(19) United States
(12) Patent Application Publication
(10) Pub. No.: US 2024/0091490 A1
(43) Pub. Date: Mar. 21, 2024

(54) INTRODUCER WITH REINFORCEMENT ELEMENT HAVING A MORE DENSE DISTAL PORTION AND RELATED SYSTEMS AND METHODS

(71) Applicant: Merit Medical Systems, Inc., South Jordan, UT (US)

(72) Inventor: Christian Lentz, Sandy, UT (US)

(21) Appl. No.: 18/463,048

(22) Filed: Sep. 7, 2023

Related U.S. Application Data
(60) Provisional application No. 63/375,022, filed on Sep. 8, 2022.
(60) Provisional application No. 63/375,926, filed on Sep. 16, 2022.

(51) Int. Cl. A61M 25/00

(52) U.S. Cl. CPC A61M 25/0012; A61M 25/0043; A61M 25/0097; A61M 2025/0063

(57) ABSTRACT

The present disclosure illustrates an introducer sheath with a braided or coiled wire frame. The wire frame of the introducer sheaths described herein include a distal portion having the wire disposed more densely than a proximal portion of the wire frame. The introducer sheaths also include a jacket encompassing the wire frame and a liner forming an inner wall.

### RELATED APPLICATIONS

This application claims priority to U.S. Provisional Application No. 63/375,022, filed on Sep. 8, 2022 and titled “Introducer With Reinforcement Element Having A More Dense Distal Portion And Related Systems And Methods” and U.S. Provisional Application No. 63/375,926, filed on Sep. 16, 2022 and titled “Introducer With Reinforcement Element Having A More Dense Distal Portion And Related Systems And Methods” which are hereby incorporated by reference in their entirety.

### TECHNICAL FIELD

The present disclosure relates generally to medical sheaths including introducer sheaths and methods to manufacture introducer sheaths. More particularly, some embodiments relate to introducer sheaths with a wire frame having a distal portion with wire disposed more densely than a proximal portion of the wire frame.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 is a perspective view of an introducer sheath with a sheath shaft including a braided wire frame, according to one embodiment.

FIG. 1A is a cross-sectional view, taken through line 1A-1A of a portion of the introducer sheath of FIG. 1.

FIG. 1B is a side view of a portion of the braided wire frame of the introducer sheath of FIG. 1.

FIG. 1C is a side view of a portion of a braided wire frame of an introducer sheath, according to one embodiment.

FIG. 2A is a perspective view of an introducer sheath with a sheath shaft including a coiled wire frame, according to one embodiment.

FIG. 2B is a side view of a portion of the coiled wire frame of the introducer sheath of FIG. 2A.

FIG. 2C is a side view of a portion of a coiled wire frame of an introducer sheath, according to an embodiment.

FIG. 2D is a cross-section view, taken through the line 2D of FIG. 2C.

FIG. 3 is a flow diagram of a method of manufacturing an introducer sheath, according to one embodiment.

FIG. 4 is a flow diagram of a method of manufacturing an introducer sheath, according to one embodiment

FIG. 5 is an exploded view of an introducer sheath with a shaft including a wire frame, according to one embodiment.

FIG. 6A is a perspective component view of an introducer sheath assembly, according to one embodiment.

FIG. 6B is a perspective view of the introducer sheath assembly of FIG. 6A, assembled for use.

FIG. 7 is a flow diagram of a method of manufacturing an introducer sheath, according to one embodiment

FIG. 8 is a flow diagram of a method of manufacturing an introducer sheath, according to one embodiment

FIG. 9 is a perspective view of an introducer sheath assembly assembled for use, according to one embodiment.

FIG. 10A is a side view of a distal portion of a sheath tube of an introducer sheath assembly, according to one embodiment.

FIG. 10B is a side view of a distal portion of sheath tube of an introducer sheath assembly, according to one embodiment.

FIG. 10C is a side view of an intermediate portion of a sheath tube of an introducer sheath assembly, according to one embodiment.

FIG. 11 is a side view of a distal portion of a sheath tube of an introducer sheath assembly, according to one embodiment.

FIG. 12 is a side view of a distal portion of a sheath tube of an introducer sheath assembly, according to one embodiment.

FIG. 13 is a cross-sectional view of a portion of a sheath shaft, according to one embodiment.

### DETAILED DESCRIPTION

This disclosure describes medical sheaths with a reinforcement element of a wire frame having a distal portion with wire disposed more densely than elsewhere on the wire frame, and methods to manufacture such an introducer sheath. For example, the wire frame may include a coiled wire frame with a distal portion having the wire coiled back over the distal portion of the coiled wire frame such that there are two portions of coiled wire overlapping one another at the distal portion. In another example, the wire frame may include a braided wire frame where the wire is braided more densely (e.g., more picks/inch) at the distal portion than elsewhere on the wire frame. According to many embodiments, the wire frame includes a radiopaque material, and the distal portion having the wire more densely disposed therein (e.g., double coiled or greater picks/inch braid) may be function as a radiopaque marker during use of the introducer sheath. For example, the distal portion of the wire frame may be used for fluoroscopy identification. While embodiments herein refer to introducer sheaths, the same features may be included on other types of sheaths such as guiding sheaths.

Introducer sheaths are used in a variety of diagnostic and therapeutic procedures to provide access to a patient's vascular system. When an introducer sheath is placed in the vasculature, the introducer sheath may facilitate exchange of guidewires, catheters, contrast media, and various fluids while providing access to the vasculature and a hemostatic seal.

To facilitate the exchange of medical tools and fluids, the introducer sheath includes a hub configured to remain exterior to the patient's skin. The hub forms a chamber that may be accessed through various ports. For example, a hub may include a side port and an introducer bore. In some embodiments, the side port is fluidly coupled to a fluid channel that is controlled by a stop-cock. The fluid channel conveys fluids or medicaments to and from the hub. A practitioner may introduce guidewires, catheters, stents, balloons, and other articles and/or materials to be introduced into the patient through the introducer bore. A valve or a seal may maintain hemostasis of the introducer sheath while allowing a medical instrument to be introduced through the introducer bore into the chamber. The hub chamber is in fluid communication with a sheath shaft. The sheath shaft is inserted into the vasculature of the patient, and provides through the skin to the vasculature.

A problem with current sheaths is vascular access bleeding that sometimes occurs after the sheath shaft has been removed. In general, there is a relationship between the size of the outer diameter of the inserted sheath shaft and the risk of bleeding complications. Thus, sheaths shafts with thinner walls correlate to a decrease in the size of the outside diameter (and therefore a decrease in the size of the hole at the vascular entry site) without decreasing the size of the inside diameter of the sheath. Thus, thin walled sheaths may reduce bleeding complications when compared to thicker wall sheaths with the same inside diameter.

However, simply reducing the thickness of the walls of a sheath shaft introduces additional concerns. Specifically, with thinner walls, there may be a greater concern of kinking and deformation from a cylindrical shape. When a sheath shaft kinks, the passageway to the vascular system of a patient may be blocked. If a sheath shaft does not maintain its shape, medical instruments may not fit.

To maintain the shape of a thin walled sheath shaft, the wall of the shaft may be reinforced. A polymer shaft may be reinforced with a metal reinforcing element, such as a braided or coiled metal frame. The reinforcing element may thus increase the strength, stiffness, burst strength, creep resistance, and other properties of the shaft.

Braided and/or coiled metal frames may also have a desirable spring temper that both resists kinks and increases the capacity of the shaft to temporarily elastically deform, then spring back without creating a permanent kink. This, in turn, reduces instances where a kinked or deformed introducer shaft must be removed and replaced during a therapy.

In certain embodiments of introducer sheaths having a braided wire frame described herein, a portion of the braided wire frame may be annealed to both reduce wall thickness and maintain a desirable wall stiffness, kink resistance, and kink recovery. Through only annealing a portion of the braided frame, the metal has a reduced tendency to unravel along the annealed portion and the braided frame maintains a desirable spring temper along the unannealed portions. In certain embodiments of introducer sheath having a coiled wire frame, the coiled wire is free from annealing.

In some embodiments, an introducer shaft is coupled to a hub at a proximal end. The shaft forms a lumen that is in fluid communication with the chamber of the hub. In some embodiments, the shaft may include a braided metal frame with an annealed distal portion and a second portion of the braided metal frame that is unannealed. The annealed distal portion reduced the tendency of the braided metal frame to unravel at a distal end. In some embodiments, the hub may prevent the braided metal frame from unraveling at the proximal end. In some embodiments, the braided metal frame may include an annealed proximal portion. In embodiments having a coiled wire frame, both the distal portion and the proximal portion of the coiled wire frame may be unannealed (e.g., free from annealing). An outer polymer member such as a jacket may encompass the braided or coiled wire reinforcing element, and an inner polymer member such as a liner may form an inside surface of the wall of the shaft.

Introducer sheaths having wire more densely disposed in the distal portion of a wire frame may be manufactured using the methods described in more detail below. In some embodiments, one or more metal wires are coiled or wound on a spring winder from a proximal end to a distal end, then further coiled or wound from the distal end only partially towards the proximal end to form the distal portion having wire more densely disposed than elsewhere in the wire frame. In some embodiments, metal wires are braided to form a frame of a sheath shaft, and the distal portion of the frame may include wires braided at a greater pick/inch than elsewhere in the frame to form the distal portion having wire more densely disposed than elsewhere in the wire frame. A liner and a jacket are reflowing or melt-bonded onto the frame, and a hub is overmolded around a proximal portion of the reflowed shaft. These methods may be done along a length sufficient to produce a plurality of introducer sheaths. For example, in some embodiments, a larger amount of wire may be braided along a nylon core to form a frame. The nylon core may be heated and stretched to reduce the diameter of the nylon core for removal of the frame. A hub may be overmolded or otherwise secured around a proximal portion of the sheath shafts.

The phrase “coupled to” is broad enough to refer to any suitable coupling or other form of interaction between two or more entities, including mechanical, fluidic and thermal interaction. Thus, two components may be coupled to each other even though they are not in direct contact with each other. The phrase “fluid communication” is used in its ordinary sense, and is broad enough to refer to arrangements in which a fluid (e.g., a gas or a liquid) can flow from one element to another element when the elements are in fluid communication with each other.

The terms “proximal” and “distal” are opposite directional terms. As used herein, the distal end of a device or component is the end of the component that is furthest from the physician during ordinary use. The proximal end refers to the opposite end, or the end nearest the physician during ordinary use. For example, the proximal end of an introducer sheath used in minimally invasive vascular treatment is the end accessible to a practitioner during use, while the distal end is disposed within a patient's vascular system when the sheath is placed into such a patient.

An assembler may be any person, system, or machine used in the manufacture of the introducer sheaths.

Embodiments may be understood by reference to the drawings, wherein like parts are designated by like numerals throughout. The components of the embodiments as generally described and illustrated in the figures herein can be arranged and designed in a wide variety of different configurations. Thus, the following more detailed description of various embodiments, as represented in the figures, is not intended to limit the scope of the present disclosure, but is merely representative of various embodiments. While various aspects of the embodiments are presented in drawings, the drawings are not necessarily drawn to scale unless specifically indicated.

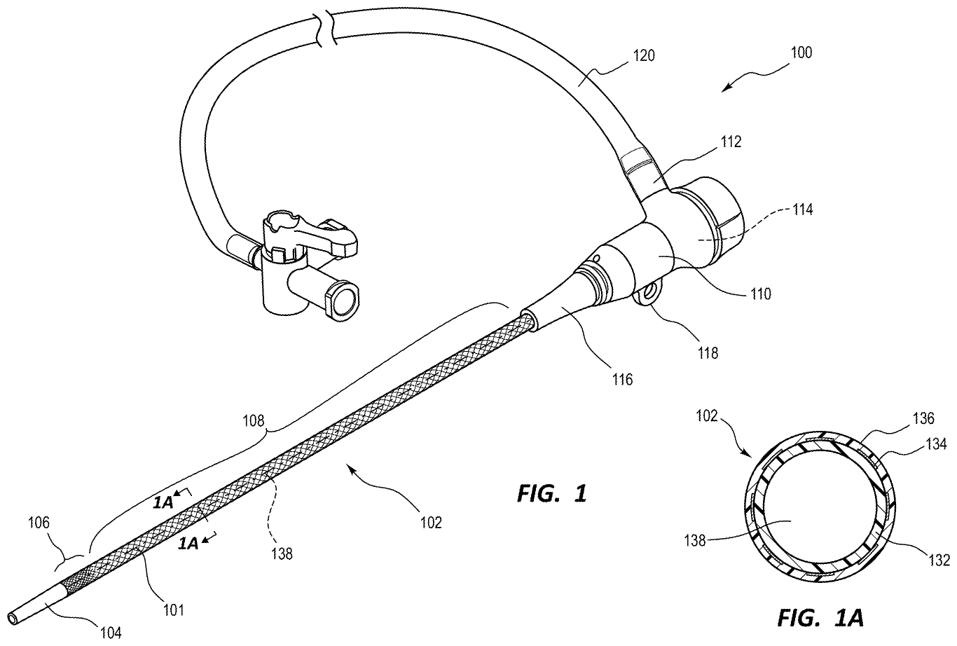

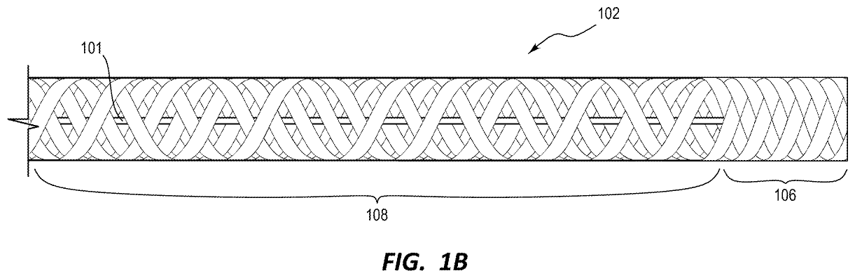

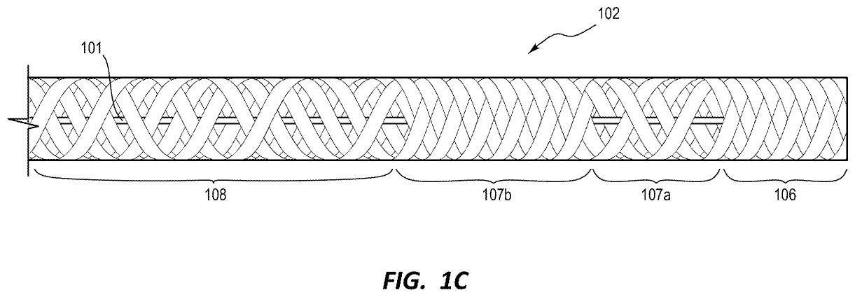

FIG. 1 is a perspective view of an introducer sheath 100 comprising a sheath shaft 102 including a braided wire frame 134 including a distal portion 106 having a greater picks/inch braid than a proximal portion 108 of the wire frame 134, according to one embodiment. As shown, the introducer sheath 100 can include the sheath shaft 102 and a hub 110. The sheath shaft 102 is coupled to and in fluid communication with the hub 110. During some procedures, the hub 110 is intended to remain exterior of a patient, and the sheath shaft 102 is intended to at least partially be placed within the vascular system of the patient.

The hub 110 forms a chamber that may be accessed via a side port 112 or an introducer bore 114. A suture ring 118 is coupled to the hub 110 and provides a mechanism allowing a practitioner to grasp the introducer sheath 100 while allowing the introducer sheath 100 to be sutured or fastened to the patient once the introducer sheath 100 has been properly placed. The side port 112 and the introducer bore 114 provide entry for medical devices and fluids. For example, a physician may insert a dilator into the introducer bore 114 to assist with placing the introducer sheath 100. The dilator enters the introducer bore 114 through a seal or a valve that maintains hemostasis when the introducer sheath 100 in in communication with the vasculature. Similarly, a fluid channel 120 may couple to the side port 112, establishing a fluid passageway with the chamber of the hub 110. In some embodiments, a sleeve may be used to swage the fluid channel 120 onto the side port 112. A sleeve 122 may be placed over the fluid channel 120 onto the side port 112. Any medical instrument or fluids that enter the chamber of the hub 110 may continue through an opening at the hub distal end 116 into a sheath shaft lumen 138. Thus, the side introducer bore 114 and a lumen of the fluid channel 120 may both be in fluid communication with the sheath shaft lumen 138.

A tip 104 is coupled to the sheath shaft 102. A cuff (not shown) may overlap the tip-shaft joint to strengthen the joint. The sheath shaft 102 may be formed from the braided metal frame 134 with an exterior and interior surface coated with a polymer material. In the illustrated embodiment, a liner 132 is coupled to the interior surface of the braided metal frame 134 and defines the inside surface of the sheath shaft 102 and a jacket 136 is coupled to the exterior surface of the braided metal frame 134 and defines the outside surface of the sheath shaft 102. FIG. 1A is a cross-sectional view, taken through line 1A-1A of a portion of the introducer sheath 100, showing elements of the sheath shaft 102. FIG. 1A may be understood as schematic in nature, though the liner 132, braided metal frame 134, and jacket 136 are shown as distinct layers, as further detailed below, the polymer materials of the liner 132 and jacket 136 may be melted and reflowed together, bonding to the braided metal frame 134 and each other and filling any openings in the braided metal frame 134.

The introducer sheath 100 may have a thinner wall when compared with traditional introducer sheaths. In some embodiments, the introducer sheath 100 may have an

$$\\ \frac{inner diameter lower limit}{outer diameter upper limit}$$

ratio of greater than 0.85. For example, an introducer sheath for a 4 French (4F) needle or catheter may have an

$$\\ \frac{inner diameter lower limit}{outer diameter upper limit}$$

ratio of greater than 0.85, an introducer sheath for a 5F needle or catheter may have an

$$\\ \frac{inner diameter lower limit}{outer diameter upper limit}$$

ratio of greater than 0.87, an introducer sheath for a 6F needle or catheter may have an

$$\\ \frac{inner diameter lower limit}{outer diameter upper limit}$$

ratio of greater than 0.89, an introducer sheath for a 7F needle or catheter may have an

$$\\ \frac{inner diameter lower limit}{outer diameter upper limit}$$

ratio of greater than 0.90. In some embodiments, the introducer sheath 100 may have an

$$\\ \frac{inner diameter}{outer diameter}$$

ratio ranging between 0.87 and 0.93. For example, an introducer sheath for a 4F needle or catheter may have an average

$$\\ \frac{inner diameter}{outer diameter}$$

ratio ranging between 0.87 and 0.89, an introducer sheath for a 5F needle or catheter may have an average

$$\\ \frac{inner diameter}{outer diameter}$$

ratio ranging between 0.89 and 0.90, an introducer sheath for a 6F needle or catheter may have an average

$$\\ \frac{inner diameter}{outer diameter}$$

ratio ranging between 0.90 and 0.91, and an introducer sheath for a 7F needle or catheter may have an average

$$\\ \frac{inner diameter}{outer diameter}$$

ratio ranging between 0.92 and 0.93.

At least a portion of the wire frame 134 may be annealed and at least a portion of the wire frame 134 may be unannealed. In some embodiments, at least some of the distal portion 106 and/or the proximal portion 108 may be annealed. For example, a distal end of the distal portion 106 and/or a proximal end of the proximal portion 106 may be annealed, while the wire frame 134 remains unannealed between the distal end and the proximal end of the wire frame 134. Various aspects and positioning of annealing a wire frame of a sheath are disclosed in U.S. patenttent Publication Ser. No. 17/457,878, the disclosure of which is incorporated herein, in its entirety, by this reference. The term unannealed refers to portions of the braided metal frame 134 that retain or otherwise are configured with more spring temper than an annealed portion. The tip 104 may be coupled to the sheath shaft 102 adjacent an annealed portion of the distal portion 106. In some embodiments, a cuff may be positioned across the joint between the tip 104 and the annealed portion of the distal portion 106. The cuff may smooth the transition between the tip 104 and the annealed portion of the distal portion 106 and increase the strength of the joint.

FIG. 1B is a side view of a portion of the braided metal frame 134 with the distal portion 106 having wire more densely disposed (e.g., closer together) than the proximal portion 108. For example, the proximal portion 108 of the wire frame 134 may be braided or woven at a first pick/inch (PPI) and the distal portion 106 of the wire frame 134 may be braided or woven at a second PPI that is greater than the first PPI of the proximal portion 108. With the distal portion 106 having a braid or weave with a greater picks/inch than the braid or weave of the proximal portion 108, the distal portion 106 includes wire more densely disposed than the proximal portion 108. The braided wire frame 134 includes one or more radiopaque strands of material may be used in the braided metal frame 134. For example one or more wires of tungsten, palladium, platinum, gold, tantalum, depleted uranium, or high radiopaque wire may be weaved into the braided metal frame for fluoroscopy identification. In many embodiments, the braided wire frame 134 consists or consists essentially of the radiopaque material. Accordingly, the distal portion 106 may include more densely disposed radiopaque strands of material than the proximal portion 108 of the braided wire frame 134. The more densely disposed radiopaque strands of material in the distal portion 106 act as a radiopaque marker in fluoroscopy identification, thereby allowing a user to more easily identify the distal region of sheath shaft 102 during use.

In some embodiments, the distal portion 108 includes a longitudinal length of less than about 125 mm, less than about 100 mm, less than about 75 mm, less than about 50 mm, less than about 25 mm, less than about 10 mm, less than about 5 mm, less than about 3 mm, less, than about 2 mm, less than about 1 mm, about 1 mm to about 125 mm, about 1 mm to about 5 mm, about 5 mm to about 10 mm, about 10 mm to about 25 mm, about 25 mm to about 50 mm, about 50 mm to about 100 mm, about 100 to about 150 mm, about 1 mm, about 2 mm, about 3 mm, about 4, about 5, about 10 mm, about 25 mm, about 50 mm, about 75 mm, or about 100 mm.

In some embodiments, the second PPI of the distal portion 106 is at least about two, about three, about four, or more times greater than the PPI of the first PPI of the proximal portion 108. For example, in some embodiments, the second PPI of the distal portion 106 may be at least about 200 PPI, at least about 250 PPI, at least about 300 PPI, at least about 350 PPI, at least about 400 PPI, at least about 450 PPI, at least about 500 PPI, about 200 PPI to about 500 PPI, about 200 PPI to about 300 PPI, about 300 to about 400 PPI, or about 400 PPI to about 500 PPI. In these and other embodiments, the first PPI of the proximal portion 108 may be less than about 200 PPI, less than about 150 PPI, less than about 100, less than about 50 PPI, about 50 PPI to about 200 PPI, about 50 PPI to about 150 PPI, or about 100 PPI to about 200 PPI.

Turning to FIG. 1C, in some embodiments, a wire frame 134′ may include multiple portions having a more dense weave (e.g., having a higher PPI) and/or multiple portions having a less dense weave (e.g., having a lower PPI). For example, the wire frame 134′ may include the distal portion 106′, a first intermediate portion 107a, a second intermediate portion 107b, and the proximal portion 108′. The first intermediate portion 107a may be disposed between (e.g. adjacent to) the distal portion 106′ and the second intermediate portion 107b, and may include a less dense weave (e.g., lower PPI) than the weave of the distal portion 106′ and the second intermediate portion 107b. The second intermediate portion may be disposed between (e.g., adjacent to) the first intermediate portion 107a′ and the proximal portion 108′, and may include a more dense weave (e.g., higher PPI) than the first intermediate portion and the proximal portion 108′.

In certain embodiments, the proximal portion 106′ and the first intermediate portion 107a (adjacent the distal portion 108) may include any of the PPIs provided above in relation to the proximal portion 106′. The proximal portion 106′ and the first intermediate portion 107a may include substantially the same (e.g. equal) PPI or different PPIs that are less than at least the second intermediate portion 107b disposed between the proximal portion 106′ and the first intermediate portion 107a. In certain embodiments, the distal portion 108′ and the second intermediate portion 107b (adjacent the proximal portion 106′) may include any of the PPIs provided above in relation to the distal portion 108′. The distal portion 108′ and the second intermediate portion 107b may include substantially the same (e.g. equal) PPI or different PP Is that are greater than at least the first intermediate portion 107a disposed between the distal portion 108′ and the second intermediate portion 107b.

In these and other embodiments, the distal portion 108′ may extend a longitudinal length of less than about 2 mm, the first intermediate portion 107a adjacent the distal portion 108′ may extend a longitudinal length of about 3 mm to about 7 mm, the second intermediate portion 107b between first intermediate portion 107a and the proximal portion 106′ may extend a longitudinal length about less than about 2 mm. In a non-limiting example, the distal portion 108′ may include a braid of about 350 PPI and extend a longitudinal length about 1 mm until the first intermediate portion 107a, the first intermediate portion 107a may include a braid of about 100 PPI that extends a longitudinal length of about 5 cm between the distal portion 108′ and the second intermediate portion′, the second intermediate portion 107b may include a braid of about 350 PPI that extends a longitudinal length of about 1 mm between the first intermediate portion 107a and the proximal portion 106′, and the proximal portion 106′ may include a braid of about 100 PPI.

In use, the distal portion 108′ and the second intermediate portion 107b having the more dense weave(s) would be visible under fluoroscopy as distinct from the first intermediate portion 107a and the proximal portion 106′ having the less dense weave(s). The distance between the distal portion 108′ and the second intermediate portion 107b (e.g., the longitudinal length of the first intermediate portion 107a) may be known by the operator and used to measure anatomical features such as a lesion or vessel aneurysm length. Any number or combination of portions with more dense weave(s) may be used. For example, a single device may have two, three, four, or more dense portions that may or may not be distributed at even distances from each other. These portions can be configured for measurement of anatomical features, identification of portions of the introducer sheath 100, or both.

The sheath shaft 102 also may include a safety line 101 extending longitudinally through the braided wire frame 134. The safety line 101 may include an axial fiber such as a wire, including solid wires and bundles of smaller fibers or filaments, and may be secured to the proximal portion 108 and the distal portion 106 of the braided wire frame 134. In some embodiments, the safety line 101 includes a tungsten wire or tungsten fiber. The safety line 101 may comprise a round cross section, rectangular, or any other shape, including a flat wire or ribbon shape. A first end of the safety line 101 may be secured (e.g., welded) to the braided wire frame 134 proximate to the proximal end of the wire frame 134, and a second end of the safety line 101 may be secured (e.g., welded) to the braided wire frame 134 proximate to the distal end of the braided wire frame 134. In some embodiments, the safety line 101 may be disposed between an interior surface of the braided wire frame 134 and the liner 132. In the unlikely event the wire frame 134 or the sheath shaft 102 breaks during use, the safety line 101 prevents the broken end of the sheath shaft 102 from being separated in the patient from the rest of the sheath shaft 102.

The distal portion 106 also may include annealed portion is located at the distal end of the distal portion. Annealing at least some of the distal portion 106 removes residual stresses and the spring temper of the braided metal frame 134 along the annealed portion of the distal portion 106. When annealed, the fibers of the annealed portion do not tend to self-straighten and unravel in the same manner as the unannealed fibers of a braided metal frame.

The presence of the annealed portion at the distal end of the distal portion 106 also helps maintain the braid along the unannealed portion of the braided metal frame 134. Though the fibers along the unannealed portion retain their spring temper, interaction with adjacent fibers and with the annealed portion mitigates the ability of these fibers to self-straighten such that they unravel. Thus, the annealed portion may prevent the remaining unannealed portion from unraveling. Thus, the partially annealed braided metal frame 134 may maintain the spring temper of an unannealed braided metal frame along the majority of its length and while still tending to remain in its tubular braided shape without outside constraints that would be required to retain the shape of a wholly unannealed braided metal frame.

In some embodiments the annealed portion at the distal end of the wire frame 134 may be approximately 1/32 inches long to approximately ¼ inches long, including about 1/16 inches long to about 3/16 inches long and about ⅛ inches long.

The braided metal frame 134 may be braided over a braiding core, according to one embodiment. The braiding core provides a surface for the wires to be braided around to form the braided metal frame 134. Various aspects and positioning of braiding a wire frame of a sheath are disclosed in U.S. patent Publication Ser. No. 17/457,878, the disclosure of which is incorporated herein, in its entirety, by this reference.

The braided metal frame 134 may be made from wires with a desired spring temper. For example, tempered steel or brass wires may have a desired spring temper induced to increase their upper limit of elasticity. In some embodiments, steels used in the braided metal frame 134 may include stainless steel, low-alloy, medium-carbon steel, nitinol, and high-carbon steel including those with high yield strength. The spring temper of the wires may allow the braided metal frame 134 to return to its original shape despite deflection, deformation, and/or twisting. In some embodiments, the braided metal frame 134 may include flat wires. In other embodiments, the braided metal frame 134 may include round wires. In yet other embodiments, the braided metal frame 134 may include a combination of flat and round wires. Each strand of the braided metal frame 134 may include one or more wires. For example, a five-strand bobbin may be used to braid a frame with five wires in every braid. The tightness of the weave of the braided metal frame may be altered to adjust the stiffness and flexibility of the introducer sheath.

The braiding core may comprise a thermoplastic that becomes pliable or moldable above a specific temperature and solidifies upon cooling. For example, in some embodiments, nylon may be extruded to form the braiding core. The diameter of the braiding core corresponds to the inside diameter of the braided metal frame 134, which thus relates (when adjusted for polymer lines and jackets) to the finished outside and inside diameters of a finished sheath shaft (such as sheath shaft 102 of FIG. 1) of an introducer sheath. When the braided metal frame 134 has been created, an assembler may heat the braiding core to a point that the thermoplastic becomes pliable, and stretch the braiding core. Stretching the braiding core causes the braiding core to neck down, resulting in a longer core with a smaller diameter. The smaller diameter allows an assembler to easily remove the braiding core from the braided metal frame 134.

After the braiding core is removed, an assembler may load the braided metal frame 134 on the annealing mandrel to anneal at least a portion of the braided metal frame 134.

In some embodiments, an assembler uses electromagnetic waves such as radio frequency (RF) or microwaves to selectively anneal portions of the braided metal frame 134. For example, RF radiation may be applied to a portion of the braided metal frame 134 to anneal the material along that portion to form an annealed portion. The braided metal frame 134 may be selectively annealed by transferring heat to the braided metal frame 134 using a heated element or a flame. The annealed portion may be disposed near the distal tip of the braided metal frame 134. The annealing process allows for crystalline restructuring between adjacent braids of the annealed portion. The crystalline restructuring of the annealed portion, such as at the intersection between adjacent braids, may tend to prevent the unannealed portion from unraveling. Additionally, annealing may reduce the tendency of the braids to straighten out, also tending to prevent unraveling.

The length of the annealed portion may vary based on application. In some embodiments, the annealed portion may be approximately 1/32 inches long to approximately ¼ inches long, including about 1/16 inches long to about 3/16 inches long and about ⅛ inches long. In some embodiments, a plurality of portions on the length of a long braided metal frame may be annealed. The long braided metal frame may subsequently be cut to form multiple braided metal frames for inclusion in introducer sheath shafts. The positions of the plurality of annealed portions along the long braided metal frame may thus ultimately correspond to portions that will be disposed as distal ends of a plurality of introducer sheath shafts.

A low surface friction mandrel may be formed from or coated with low friction materials to ease loading and removal of the liner and braided metal frame 134. For example, in some embodiments, the low surface friction mandrel may be a polytetrafluoroethylene (PTFE) or PTFE-coated mandrel to slide a stainless steel metal frame onto. In some embodiments, lubricants may be used to reduce the friction on the surface of the low surface friction mandrel.

The liner material may comprise polyamide resins configured to seal the interior surface of the braided metal frame 134. The liner 132 can include a surface comprising a lubricious polymeric material. For example, the material can comprise any bio-compatible material having low frictional properties (e.g., TEFLON®, PTFE, fluorinated ethylene propylene (FEP), polyethylene, polyamide, ethylene chlorotrifluoro-ethylene, ethylene tetrafluoroethylene, PVDF).

When the braided metal frame 134 is slid over the liner 132, the fibers of the braided metal frame 134 may tend to bunch forming a bunched section. Such bunching may result in the braided metal frame 134 becoming non-uniform. Non-uniformity, in turn, may cause inconsistencies along the surface of the introducer sheath shaft, and alter the flexibility along the introducer sheath shaft. Therefore, in some embodiments, the braided metal frame 134 may be stretched after being placed on the liner 132 to restore uniformity. In some embodiments, the braided metal frame 134 may be braided directly onto the mandrel and annealing may occur while braiding. This may prevent bunching of the braided metal frame 134.

An assembler may stretch the braided metal frame 134 until it is approximately uniform in the proximal portion 108. A proximal portion may be twisted down against the liner 132 and low surface friction mandrel after stretching. The twisting may secure the proximal portion in place and cause the remainder of the braided metal frame 134 to maintain its shape and position. The uniformity of the braided metal frame 134 results in a similar flexibility, kink resistance, and diameter along the entire length of the introducer sheath shaft. The step of stretching the braided metal frame 134 to restore uniformity may not be needed in instances wherein the braided metal frame 134 is slid over the liner 132 in such a manner as to retain its uniform consistency.

The braided metal frame 134 and the liner 132 may be loaded on the low surface friction mandrel, with a jacket disposed over the braided metal frame 134 and an FEP shell disposed over the jacket 136. The jacket 136 may have a hydrophilic coating and a larger diameter than the braided metal frame 134. The FEP shell may have a larger diameter than the jacket 136 and encompass the jacket 136, braided metal frame 134, and liner 132.

All of the components may be heated to melt or reflow the liner 132 and the jacket 136. Heating the FEP shell causes it to shrink, reducing its diameter. The reduced diameter of the FEP shell causes the sidewalls of the FEP shell to apply pressure to the encompassed elements (i.e., the jacket 136, braided metal frame 134, and liner 132). The reflow temperature is within a range that causes the jacket 136 and liner 132 to melt, but not the FEP shell. Thus, the jacket 136 and liner 132 are reflowed to the metal frame 134 to create a composite conduit forming a sheath shaft 102.

The sheath shaft 102 may be loaded on a cutting mandrel. The distal and proximal ends of the sheath shaft 102 may be cut and removed. In some embodiments, part of the annealed portion is removed, ensuring that the distal tip of the braided metal frame 134 comprises an edge of the annealed portion.

The sheath shaft 102 may include the tip 104 coupled to the composite conduit sheath shaft 102 adjacent the distal tip of the braided metal frame 134. The tip 104 may comprise a polymeric material configured to be malleable to reduce trauma when the introducer sheath enters the vascular system and increase trackability of the introducer over a guidewire. A butt joint may couple the tip 104 to the sheath shaft 102. A butt joint ensures smoothness through the transition from the tip 104 to the sheath shaft 102. In some embodiments, the tip 104 may overlap the sheath shaft 102. In some embodiments, a cuff may overlap the tip 104 and the annealed section of the sheath shaft 102 to strengthen the butt joint. Coupling the tip 104 to the sheath shaft 102 may be accomplished, for example, via loading the components on a low surface friction mandrel (not necessarily the same low surface friction mandrel discussed above, though it may be). An FEP shell (not necessarily the same FEP shell discussed above) may encompass the tip 104 and the sheath shaft 102. Heat may cause the FEP shell to shrink and apply pressure while the tip 104 melts to the sheath shaft 102.

The sheath shaft 102 with an overmolded tip 104. The tip 104 may couple to the sheath shaft 102 at a distal end and may be coupled via an overmolding process. Similarly, the hub (not shown) may be overmolded at a proximal end of the sheath shaft 102. In some embodiments the tip 104 may be more radiopaque than other components to facilitate imaging.

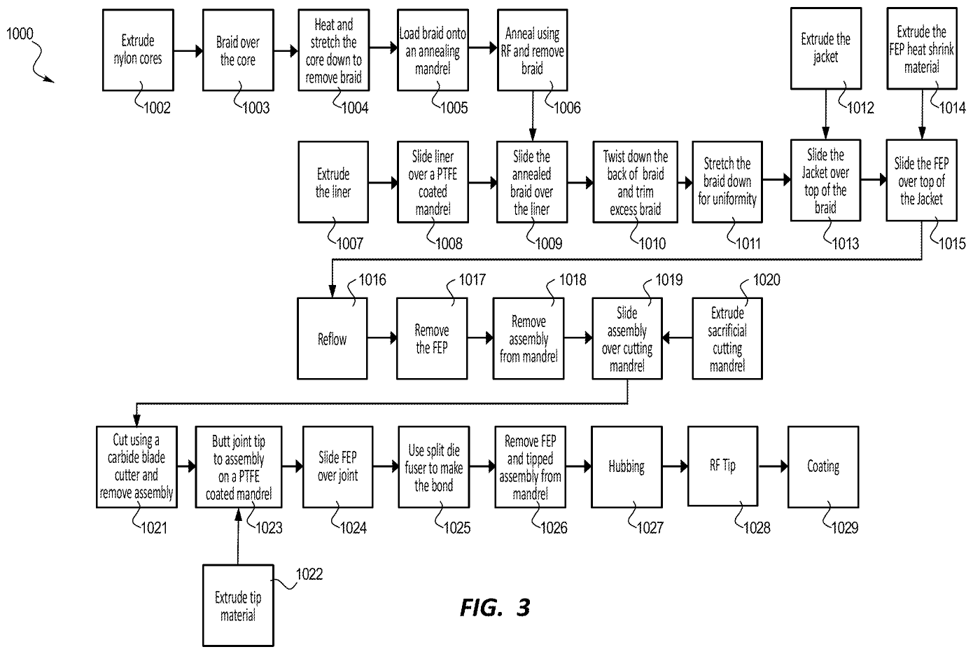

Turning ahead in the drawings, FIG. 3 is a flow diagram of an example of a method 300 of manufacturing an introducer sheath, according to one embodiment. The steps shown in FIG. 3 may be optional and not necessarily including in each process. Further, various steps may be completed in different sequences from those shown in FIG. 3.

In the illustrated process of FIG. 3, an assembler extrudes 302 a nylon core and braids 303 a metal frame over the core. In many embodiments, braiding 303 a metal frame over the core includes braiding metal wires at a first picks/inch over the core to form the proximal portion of the metal frame and braiding the metal wires over the core at a second picks/inch that is greater than the first picks/inch to form the distal portion having the wire disposed more densely than in the proximal portion. The assembler then heats and stretches 304 the core and removes the braid. The assembler loads 305 the braid onto an annealing mandrel. RF anneals 306 a portion of the braid.

A liner is extruded 307 and slides 308 over a PTFE-coated mandrel. The assembler slides 309 the annealed braid over the liner. The back of the braid is twisted 310 down, and any excess braid is trimmed. The assembler stretches 311 the braid for uniformity. The assembler extrudes 312 a jacket, and extrudes 314 an FEP heat shrink shell. The assembler slides 313 the extruded jacket over top of the braid, and slides 315 the FEP shell over the jacket. The assembly is exposed to heat, causing the liner and the jacket to reflow 316.

The FEP shell is removed 317, and the assembly is removed 318 from the mandrel. The assembler extrudes 320 a sacrificial cutting mandrel and slides 319 the assembly over the cutting mandrel. The assembler uses a carbide blade to cut 321 the ends of the assembly. The assembler extrudes 322 a tip and joins 323 the assembly to the tip using a butt joint. The assembler may place a cuff overlapping 330 the butt joint for additional strength. The assembler slides 324 an FEP shell over the joint and uses a split die fuser to bond 325 the tip to the assembly. The assembler removes 326 the assembly from the FEP shell and the mandrel and overmolds a hub 327 and a radiopaque tip 328. The assembler coats 329 the assembly with a hydrophilic coating.

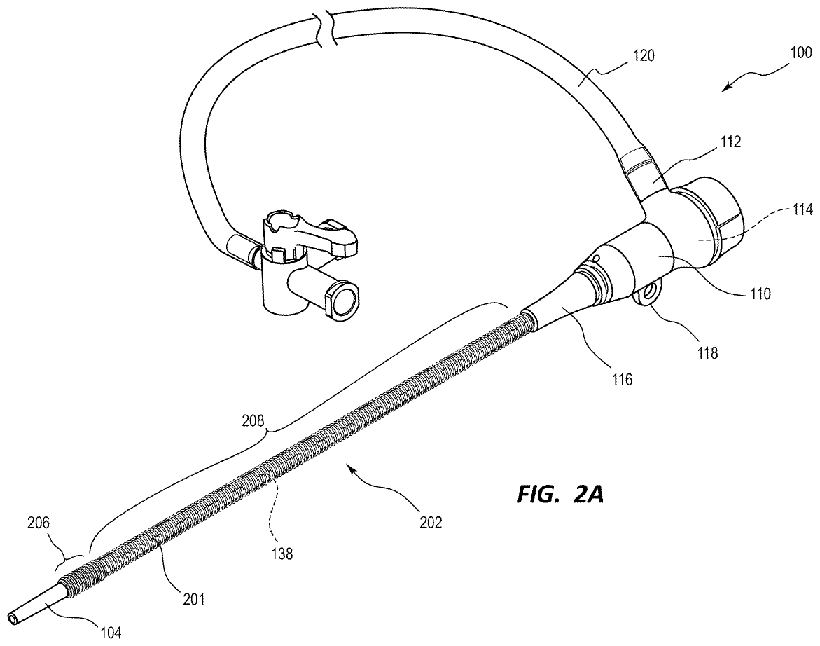

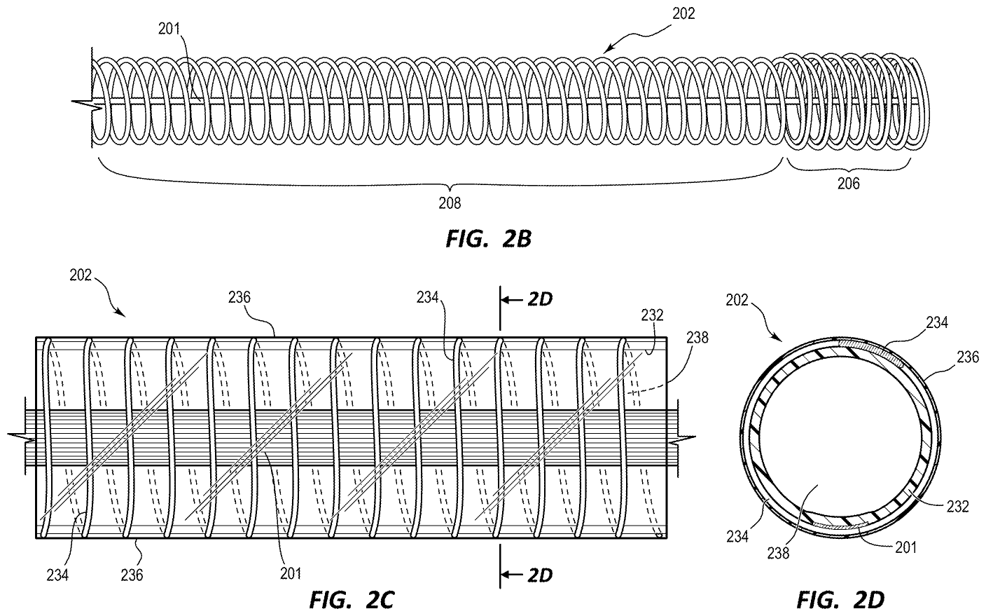

Turning back in the drawings to FIGS. 2 and 2A, in some embodiments an introducer sheath 200 includes a coiled wire (e.g. metal) frame 234 having a distal portion 206 and a proximal portion 208, with wire of the wire frame 234 being disposed more densely in the distal portion 206 than the proximal portion 208. The wire frame 234 may be disposed between a liner 232 and a jacket 236. Unless otherwise noted, the coiled wire frame 234 may include any aspect (e.g. materials or shape) of the braided wire frame 134. Accordingly, like features are designated with like reference numerals, with the leading digits incremented to “2.” For example, the embodiment depicted in FIGS. 1, 1A, and 1B includes a proximal portion 108 that may, in some respects, resemble the proximal portion 208 of FIGS. 2 and 2A. Similarly, the embodiment depicted in FIGS. 1, 1A, and 1B includes a distal portion 106 that may, in some respects, resemble proximal portion 206 of FIGS. 2 and 2A. Relevant disclosure (e.g., materials, dimensions, features) set forth above regarding similarly identified features thus may not be repeated hereafter. Moreover, specific features of the introducer sheath 200 and related components shown in FIGS. 2 and 2A may not be shown or identified by reference numerals in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the embodiments of FIGS. 2 and 2A. Any suitable combination of the features, and variations of the same, described with respect to the introducer sheath 100 and related components illustrated in FIGS. 1, 1A, and 1B can be employed with the embodiments and related components of FIGS. 2 and 2A, and vice versa. This pattern of disclosure applies equally to further embodiments depicted in subsequent figures and described hereafter, wherein the leading digits may be further incremented.

In some embodiments, the distal portion 206 of the coiled wire frame 234 includes at least two overlapping coils of wire and the proximal portion 208 of the coiled wire frame 234 includes only a single coil of wire (e.g., the proximal portion 208 does not include overlapping coils of wire). The at least two overlapping coils of wire in the distal portion 206 relative to only a single coil of wire in the proximal portion 208 results in the wire of the coiled wire frame 234 being more densely disposed in the distal portion 206 than the proximal portion 208 of the coiled wire frame 234. This more densely disposed wire in the distal portion 206 provides the same or similar radiopaque features for fluoroscopic identification as described above in relation to the distal portion 106 of the introducer sheath 100.

In some embodiments, the distal portion 206 includes a first portion of coiled wire extending a length of the distal portion 206 and a second portion of coiled wire extending the length of the distal portion such that the second portion of coiled wire overlaps the first portion of coiled wire. The first portion of coiled wire in the distal portion 206 may extend continuously to the second portion of coiled wire in the distal portion 206. For example, the first portion of coiled wire may extend from a first end of the distal portion 206 to a distal end of the distal portion 206, and the second portion of coiled wire is connected to the first portion of coiled wire at the distal end and then extends from the distal end to the first end of the distal portion. In these and other embodiments, the second portion of coiled wire may include a terminating end at the first end of the distal portion of the coiled wire frame 234. Said another way, the coiled wire may extend from the proximal portion 208 to a distal end at the distal portion 206, then double back over the distal portion 206 and terminate between the distal end and the proximal end of the coiled wire frame 234. The distal portion 206 of the wire frame 234 may extend from the distal end of the wire frame 234 to the terminating end of the wire between the distal end and the proximal end of the coiled wire frame 234. Formation of the coiled wire frame 234, then, is a continuous process that does not require a stopping and restarting of coiling the wire around, for example, a spring winder.

Doubling backing of the wire in the distal portion 206 prevents the coil from springing open. Accordingly, in many embodiments, the coiled wire frame 234 is free from annealing (e.g., annealing is absent from the coiled wire frame 234). In many embodiments, the wire in the coiled wire frame 234 includes a round wire. In other embodiments, the wire of the coiled wire frame 234 includes a flat wire. In yet other embodiments, the coiled metal frame 234 may include a combination of flat and round wires.

The sheath shaft 202 also may include the safety line 101 extending longitudinally through the coiled wire frame 234. The safety line 101 may be tungsten and may be secured to the proximal portion 208 and the distal portion 206 of the coiled wire frame 234. For example, a first end of the safety line 101 may be secured (e.g., welded) to the coiled wire frame 134 proximate to the proximal end of the wire frame 234, and a second end of the safety line 101 may be secured (e.g., welded) to the coiled wire frame 234 proximate to the distal end of the coiled wire frame 234. In some embodiments, the safety line 101 may be disposed between an interior surface of the coiled wire frame 234 and the liner 132. In the unlikely event the wire frame 234 or the sheath shaft 202 breaks during use, the safety line 101 prevents the broken end of the sheath shaft 202 from being separated in the patient from the rest of the sheath shaft 202.

Turning now to FIGS. 2B and 2C, some embodiments of the shaft 202 may include a safety line 201 include a plurality of wires or fibers bound or otherwise secured together and extending longitudinally through the coiled wire frame 234. Although the safety line 201 including a plurality of wires or fibers bound or otherwise secured together is shown with the coiled wire frame 234, the safety line 201 may be included with any wire frame described herein, such as the wire frame 134 or the wire frame 134′. The safety line 201 may include a plurality of tungsten or aromatic polyamide (e.g., aramid) fibers or other materials including composites of tungsten and/or aromatic polyamides. The plurality of fibers of the safety line 201 may be generally aligned next to one another such that the fibers are not twisted or braided with one another. As shown in FIG. 2C, the collection of individual fibers that make up the plurality of fibers in the safety line are distributed around a portion of the liner 232. Moreover, the plurality of fibers of the safety line 201 may collectively be compressed between the coiled wire frame 234 and the liner 232. Accordingly, a cross-section of the safety line 201 may be generally arcuate complementary to the coiled wire frame 234 and/or the liner 232, or may be generally planar or flat.

The safety line 202 may be secured to the proximal portion 208 and the distal portion 206 of the coiled wire frame 234. For example, a first end of the safety line 201 may be secured (e.g., welded) to the coiled wire frame 234 proximate to the proximal end of the wire frame 234, and a second end of the safety line 201 may be secured (e.g., welded) to the coiled wire frame 234 proximate to the distal end of the coiled wire frame 234. In some embodiments, the safety line 201 may be disposed between an interior surface of the coiled wire frame 234 and the liner 232. In the unlikely event the wire frame 234 or the sheath shaft 202 breaks during use, the safety line 201 prevents the broken end of the sheath shaft 202 from being separated in the patient from the rest of the sheath shaft 202.

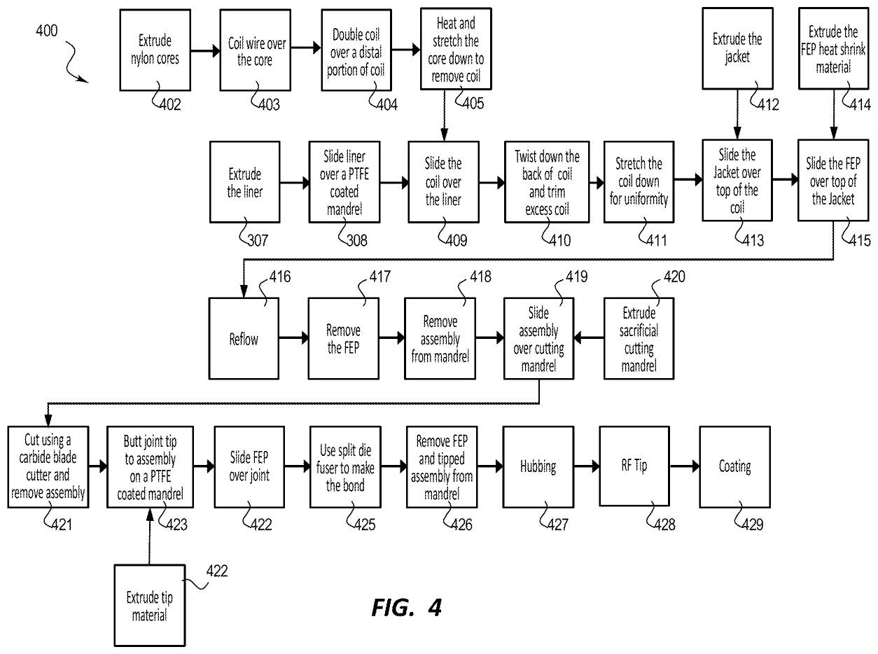

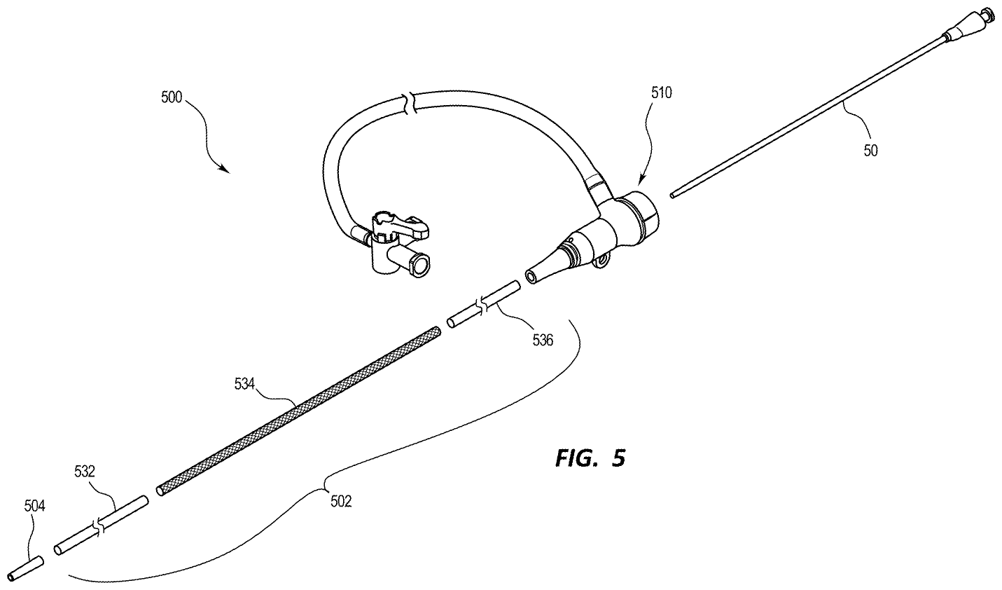

Turning ahead in the drawings, FIG. 4 is a flow diagram of an example of a method 400 of manufacturing an introducer sheath, according to one embodiment. The steps shown in FIG. 4 may be optional and not necessarily including in each process. Further, various steps may be completed in different sequences from those shown in FIG. 4.

Unless otherwise noted, the method 400 may include any aspect of the method 300. Accordingly, like steps are designated with like reference numerals, with the leading digits incremented to “4.” Relevant disclosure set forth above regarding similarly identified features thus may not be repeated hereafter. Any suitable combination of the features, and variations of the same, described with respect to the method 300 and related components can be employed with the embodiments and related components of the method 400, and vice versa. This pattern of disclosure applies equally to further embodiments depicted in subsequent figures and described hereafter, wherein the leading digits may be further incremented.

In the illustrated process of FIG. 4, an assembler extrudes 402 a nylon core and coils 403 a metal frame over the core. In some embodiments, the metal frame may be coiled by winding the metal (e.g., wire) on a spring winder. The method also may include doubling 404 the coil over a distal portion of the coil to form a distal portion having the metal disposed more densely than in the rest (e.g., proximal portion) of the metal frame. In some embodiments, the acts 403 and 404 include winding the metal on a spring winder from the proximal portion to the distal portion, and then winding the wire over the distal portion again such that the wire is disposed more densely in the distal portion than in the proximal portion. The metal, then, may extend continuously from the proximal portion to a distal end of the distal portion of the metal frame opposite to the proximal portion, and from the distal end to a terminating end of the metal at a first end of the distal portion between distal end and the proximal portion. In some embodiments, the assembler may then heat and stretch 405 the core and removes the coiled metal frame. In some embodiments, the coiled metal frame may be formed without formation and removal of the nylon core, and may be instead before formed on the spring winder, as described above.

A liner is extruded 407 and slides 408 over a PTFE-coated mandrel. The assembler slides 409 the coiled metal frame over the liner. In some embodiments, the back of the coiled metal frame may be twisted 410 down, and any excess coil at the proximal end may be trimmed. The assembler stretches 411 the coiled metal frame for uniformity. The assembler extrudes 412 a jacket, and extrudes 414 an FEP heat shrink shell. The assembler slides 413 the extruded jacket over top of the coiled metal frame, and slides 415 the FEP shell over the jacket. The assembly is exposed to heat, causing the liner and the jacket to reflow 416.

The FEP shell is removed 417, and the assembly is removed 418 from the mandrel. The assembler extrudes 420 a sacrificial cutting mandrel and slides 419 the assembly over the cutting mandrel. The assembler uses a carbide blade to cut 421 the ends of the assembly. The assembler extrudes 422 a tip and joins 423 the assembly to the tip using a butt joint. The assembler may place a cuff overlapping 430 the butt joint for additional strength. The assembler slides 424 an FEP shell over the joint and uses a split die fuser to bond 425 the tip to the assembly. The assembler removes 426 the assembly from the FEP shell and the mandrel and overmolds a hub 427 and a radiopaque tip 428. The assembler coats 429 the assembly with a hydrophilic coating.

FIG. 5 is an exploded view of an introducer sheath 500 having a sheath shaft 502, according to an embodiment. The sheath shaft 502 includes one of the braided wire frame 134 or the coiled wire frame 234 having wire disposed more densely in a distal portion of the wire frame than a proximal portion of the wire frame. A hub 510 may be secured to the sheath shaft 502 to provide access to the sheath shaft 502 for medical instruments such as a dilator 50. Further, the sheath shaft 502 may comprise a liner 532, braided metal frame 534, jacket 536, and cuff 524. The sheath shaft 502 elements may be coupled via a reflow process that melt-bonds the elements together. A tip 504 is coupled to the sheath shaft 502.

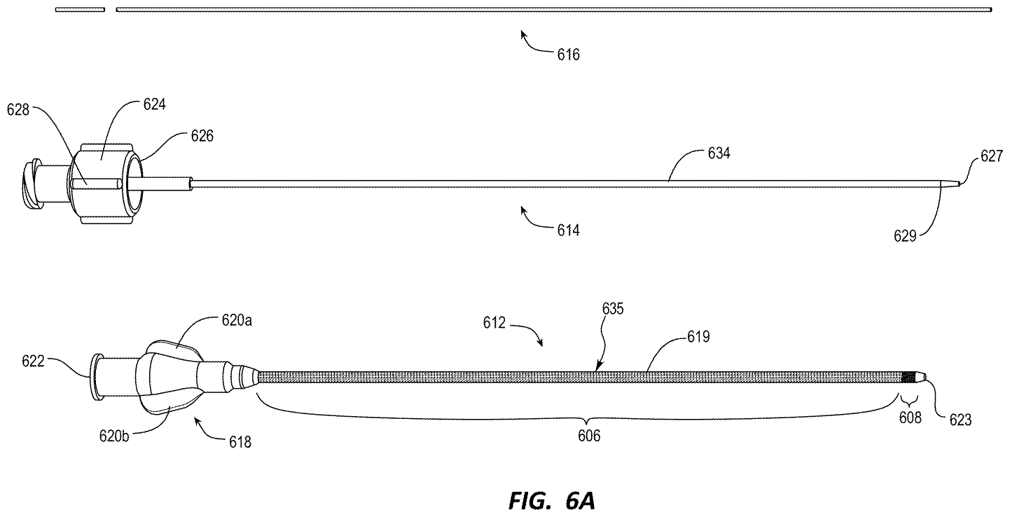

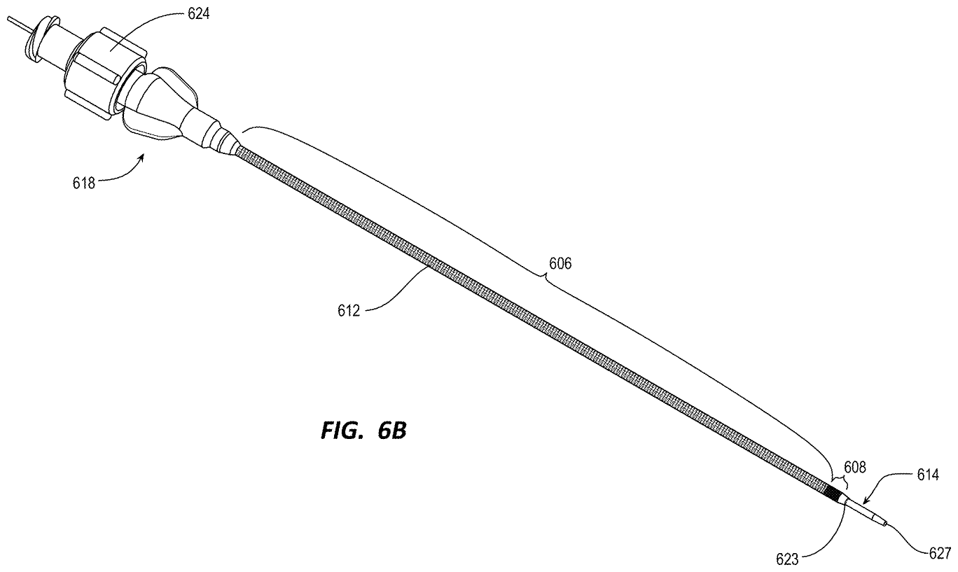

Other embodiments of introducer sheaths including a shaft having a braided or coiled wire frame with wire disposed more densely in the distal portion than the proximal portion of the wire frame are also described herein. FIG. 6A is a perspective view of an introducer sheath assembly 600 having a catheter sheath 612, a dilator 614, and a guidewire 616. The catheter sheath 612 includes a sheath tube 619 with a braided or coiled wire frame 635 disposed therein. For example, the catheter sheath 612 may include the braided or coned wire, frame 635 disposed between a liner and a jacket. Unless otherwise noted, the coiled wire frame 635 may include any aspect (e.g. materials or shape) of the braided wire frame 134, 534 or the coiled wire frame 234, and/or described in the methods 300, 400. Furthermore, the liner and the jacket of the catheter sheath 612 may include any aspect (e.g., materials or shape) of the liner 132, 232 and/or the jacket 136, 236, and/or described in the methods 300, 400.

In some embodiments, the braided or coiled wire frame 635 includes wire disposed more densely in the distal portion 608 than the proximal portion 606 of the wire frame 635. The wire frame 635, including the distal portion 608 and the proximal portion 606, may include any aspect of other wire frames described herein, such as the materials, dimensions, configurations, and positioning of: the braided wire frame 134 including the proximal portion 106 and the distal portion 108; the braided wire frame 134′ including the proximal portion 106′, the distal portion 108′, the first intermediate portion 107a, and the second intermediate portion 107b; or the coned wire frame 234 including the distal portion 208 and the proximal portion 206. Accordingly, although the wire frame 635 is shown as a coned, in other embodiments, the wire frame 635 may be braided. Furthermore, the introducer sheath assembly 600 also may include a safety line 101 or 201.

The introducer sheath assembly 600 is utilized to facilitate the introduction of a guidewire or catheter into the vasculature or other body cavity of a patient. Micropuncture catheters have been developed to provide a relatively smaller access puncture into the vasculature of the patient. Micropuncture introducer sheath assemblies include an introducer sheath which allows larger diameter guidewires to be introduced into the vasculature of the patient through the smaller access opening.

In the illustrated embodiment, introducer sheath assembly 600 comprises a catheter sheath 612, a dilator 614, and a guidewire 616. During a procedure in which introducer sheath assembly 600 is utilized, catheter sheath 612 and dilator 614 will typically be coupled together allowing the introducer sheath assembly 10 to be threaded into the patient. Dilator 614 is inserted along the length of catheter sheath 612 to provide rigidity and stiffness to facilitate the insertion of catheter sheath 612 into the patient. To Insert catheter sheath 612 and dilator 614 into the patient, guidewire 616 is first inserted into the desired position within the patient through an access needle which has been inserted through the skin of the patient and into a vein, artery, or body cavity. Once guidewire 616 has been threaded through the access needle and into the patient, the needle can be withdrawn leaving guidewire 616 in place within the patient.

The relatively small diameter of the components of a micropuncture introducer sheath assembly 600 allow for a relatively small access puncture into the patient. For example, typically the access needle utilized with guidewire 616 can be a 21 gauge access needle or smaller. This allows a smaller guidewire 616 to be utilized. For example, guidewire 616 can be a 0.018″ guidewire or smaller. By utilizing such small access needles and guidewires with introducer sheath assembly 600, the original access puncture to the patient's skin, body cavity, or vasculature is quite small. The small access puncture facilitates a shorter recovery time, less trauma to the patient, and while abbreviating the length of bleeding from the access puncture and/or the vasculature of the patient.

Once guidewire 616 is positioned in the desired location within the patient, dilator 614 is threaded over guidewire 616. As previously discussed, dilator 614 is positioned within catheter sheath 612 such that when guidewire 616 is threaded along the length of dilator 614, guidewire 16 is also threaded along the length of catheter sheath 612. Dilator 614 and catheter sheath 612 are then advanced along the length of guidewire 616 through the access puncture in the patients skin and into the desired position within the patient's body. The configuration of the tip of catheter sheath 612 and dilator 614 result in little tearing or trauma at the access puncture, Instead, catheter sheath 612 and dilator 614 results in stretching of the access puncture in a manner such that the size of the access puncture quickly returns to the original puncture size allowing for quicker healing at the access puncture site. Once dilator 614 and catheter sheath 612 have been inserted into the patient, guidewire 616 is withdrawn from the patient. Subsequently, dilator 614 will be withdrawn from the catheter sheath 612 allowing for the practitioner to access the body cavity or vasculature of the patient through catheter sheath 612.

In one embodiment, a larger diameter guidewire can be threaded through catheter sheath 612 and into the vasculature of the patient without requiring a larger access puncture typically required when a larger guidewire is inserted directly through an access needle. For example, such larger diameter guidewires can be a 0,038″ diameter guidewire which requires an approximately 0.040″ diameter puncture needle to be inserted into the patient. Utilizing a 0.040″ diameter puncture needle instead of the exemplary 21 gauge needle which is utilized in connection with catheter sheath 612 results in an access puncture that can be more than twice the size of the puncture required when utilizing introducer sheath assembly 600.

In the illustrated embodiment, catheter sheath 612 comprises a sheath hub 618 and a sheath tube 619. Sheath tube 619 is coupled to sheath hub 618 in a manner that allows for desired operation of sheath tube 619 relative to sheath hub 618. Additionally, sheath tube 619 is in fluid communication with sheath hub 618 allowing access to sheath tube 619 along the length of sheath hub 618.

In the illustrated embodiment, sheath hub 618 comprises winos 620 a, b and a lower coupler 622. Wings 620 a, b facilitates manipulation of catheter sheath 612 and introducer sheath assembly 600 by providing a gripping point for the practitioner during utilization of the introducer sheath assembly 600. Lower coupler 622 is positioned at the proximal end of catheter sheath 612. Lower coupler 622 allows for coupling of the dilator 614 to the catheter sheath 612 during operation of the introducer sheath assembly.

Sheath tube 619 includes a sheath tip 623 positioned at the distal end of the sheath tube 619. Sheath tip 623 is slightly tapered allowing for insertion of the catheter sheath into the access puncture. Sheath tube 619 typically has a resilient and somewhat flexible configuration allowing for introduction of larger diameter guidewires along the length of catheter sheath 612. The resilient nature of sheath tube 619 also permits expansion of the access puncture in the patient's skin while protecting the patient from damage as the guidewire is inserted.

Dilator 614 may comprise a dilator hub 624 and a stiffener tube. Stiffener tube provides additional rigidity and strength to dilator sleeve 634 as introducer sheath assembly 600 is inserted into the patient. Dilator hub 624 allows for manipulation of stiffener tube while also allowing for coupling of the dilator 614 to the catheter sheath 612 during utilization of the introducer sheath assembly 600. In the illustrated embodiment, dilator hub 624 comprises a lower coupler 626 and gripping members 628. Lower coupler 626 is positioned on the distal side of dilator hub 624 allowing for mating engagement of dilator hub 624 and sheath hub 618. Gripping members 628 are positioned on the outside diameter of dilator hub 624 allowing for gripping of the dilator hub 624 by the practitioner. By gripping the dilator hub 624, the practitioner can manipulate the dilator 614 to secure the dilator hub 624 to the sheath hub 618. Additionally, gripping members 628 of dilator hub 624 allow the practitioner to manipulate the introducer sheath assembly 600 during the course of the procedure.

Dilator sleeve 634 includes a dilator tip 627. Dilator tip 627 comprises a somewhat resilient member which is configured to prevent damage to the patient tissue as introducer sheath assembly 600 is threaded along the length of guidewire 616 and into the patient. Dilator tip 627 is somewhat more resilient and deformable than stiffener tube. This is due to the fact that the stiffener tube is positioned within dilator sleeve 634 but does not extend into dilator tip 627.

Transition 629 represents the point at which the stiffener tube terminates and dilator tip 627 begins. In the illustrated embodiment, transition 629 is positioned at a point that is more proximal than traditional transition points on stiffened micropuncture catheters. This allows for transition 629 to be positioned proximally to the sheath tip 623 of catheter sheath 612 when dilator 614 is threaded along the length of catheter sheath 612 and dilator hub 624 is coupled to sheath hub 618. Additionally, dilator tip 627 has a greater length from transition 629 to the distal tip of dilator tip 627. The greater length of dilator tip 627 allows a portion of dilator tip 627 to be positioned proximally to sheath tip 623 and within the catheter sheath 612. Additionally, a portion of dilator tip 627 can be positioned outside of catheter sheath 612 and distally to sheath tip 623.

The positioning of transition 629 within catheter sheath 612 allows catheter sheath 612 to provide strain relief to dilator tip 627 subsequent to lateral movement of the dilator tip 627. Positioning transition 629 within catheter sheath 612 relieves the strain that would normally be carried primarily at transition 629. Such strain is caused due to the stiffer configuration of dilator sleeve 634, which is co-extensive with the stiffener tube, and the more flexible nature of dilator tip 627. By providing strain relief subsequent to lateral movement of dilator tip 627 potentially damaging forces at transition 629 are dissipated. By dissipating such forces, kinking, buckling, or bending of catheter tip 627 at transition 629 is minimized in a manner that could result in the failure of dilator tip 27 during the procedure. In other words, minimizing the potential for damage at transition 629 provides for continued integrity of dilator 614 during the course of an insertion procedure.

As will be appreciated by those skilled in the art, a variety of types and configurations of introducer sheath assemblies can be utilized without departing from the scope or spirit of the present invention. For example, in one embodiment, the transition point is positioned at a traditional location along the length of the dilator. An elongate catheter sheath is provided such that the sheath tip is positioned distally to the transition. In another embodiment, a standard sized catheter sheath is utilized with a dilator having a shorter stiffener. An elongated dilator tip is provided such that the transition is moved proximally behind the tip of the catheter sheath. In yet another embodiment, a combination of an elongated catheter sheath, an elongated dilator tip, and a proximally positioned transition is utilized to provide a strengthened dilator tip. In yet another embodiment, the dilator hub is secured to the sheath hub utilizing other than a lower coupling. In yet another embodiment, a single hub is provided instead of two hubs.

FIG. 6B is a perspective view of an introducer sheath assembly according to one embodiment of the present invention. In the illustrated embodiment, dilator 614 is threaded along the length of catheter sheath 612. Dilator hub 624 is secured to sheath hub 618 such that catheter sheath 612 and dilator 614 operate as a single insertion instrument in cooperation with a guidewire 616. As previously discussed, a practitioner grasps one or both of sheath hub 618 and dilator hub 624 to guide dilator tip 627 over the guidewire 616 and into the insertion point as desired during the procedure.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

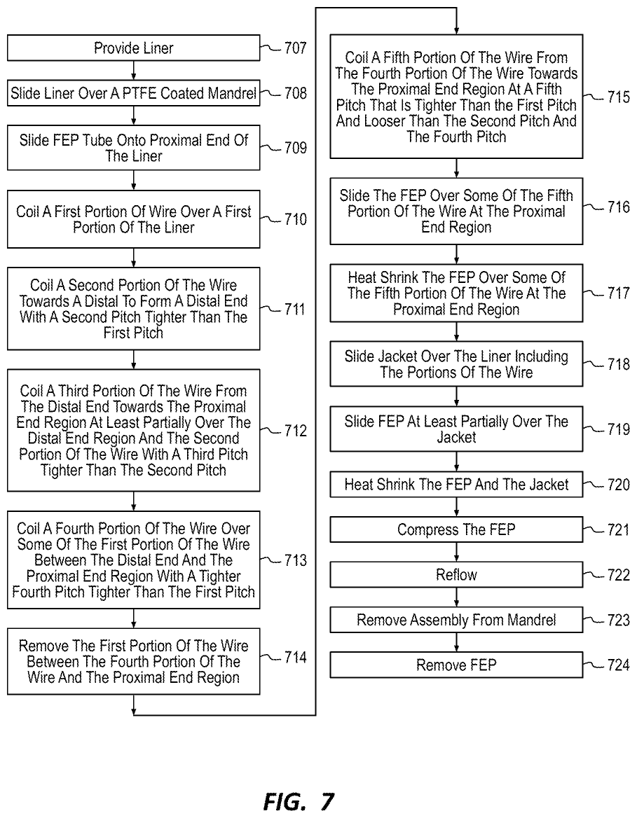

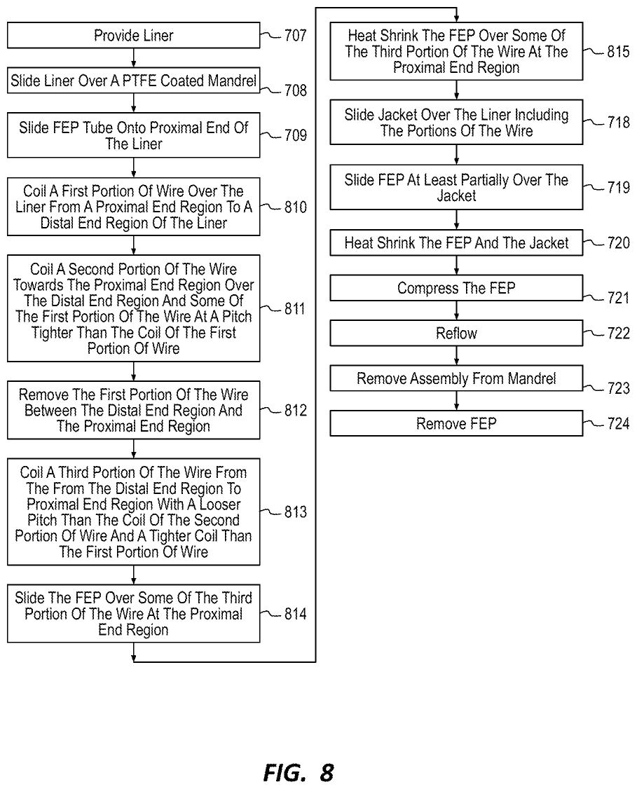

Turning now to FIGS. 7-12, in many embodiments, an introducer sheath may include a sheath tube or a sheath shaft having a wire frame that is at least partially coiled over itself or coiled over a braided portion to form distal portion or region having wire disposed more densely than elsewhere (e.g., the proximal portion) of the sheath tube the sheath shaft. For example, in some embodiments, during formation of the coiled wire frame, the wire may be coiled back over at least a portion of an initial coil around the liner. In many embodiments, at least a portion of the initial coil around the liner may be removed after the coil wire frame has been doubled over onto the initial coil or other coils to prevent unraveling of the initial coil. Accordingly, in many embodiments, the coiled wire frame may include a continuous wire coiled at varying pitches to form an identifiable distal end having wire disposed more densely therein. The sheath tubes or sheath shafts shown and described in relation to FIGS. 7-12 may be incorporated with any of the introducer sheaths described herein, such as the introducer sheaths 100, 200, 500, 600.

Formation of sheath tubes or sheath shafts may generally include providing a liner having a proximal region and a distal region, forming a coiled wire frame over the liner, and securing a jacket over the coiled wire frame and the liner. Forming the coiled wire frame over the liner in these and other embodiments may include coiling wire on the liner, coiling the wire over at least some of the wire at the distal region, removing at least some of the wire coiled on the liner between the distal region and the proximal region; and coiling the wire from the distal region towards the proximal region at a pitch that is looser than the wire coiled at the distal region. FIGS. 7 and 8 are flow diagrams of methods 700, 800 of manufacturing an introducer sheath, according to some embodiments. The methods 700, 800 may incorporate or include any aspect and/or act of the methods 300, 400 described above in greater detail. Likewise, the methods 300, 400 may include any aspect and/or act of the methods 700, 800 described in greater detail below.

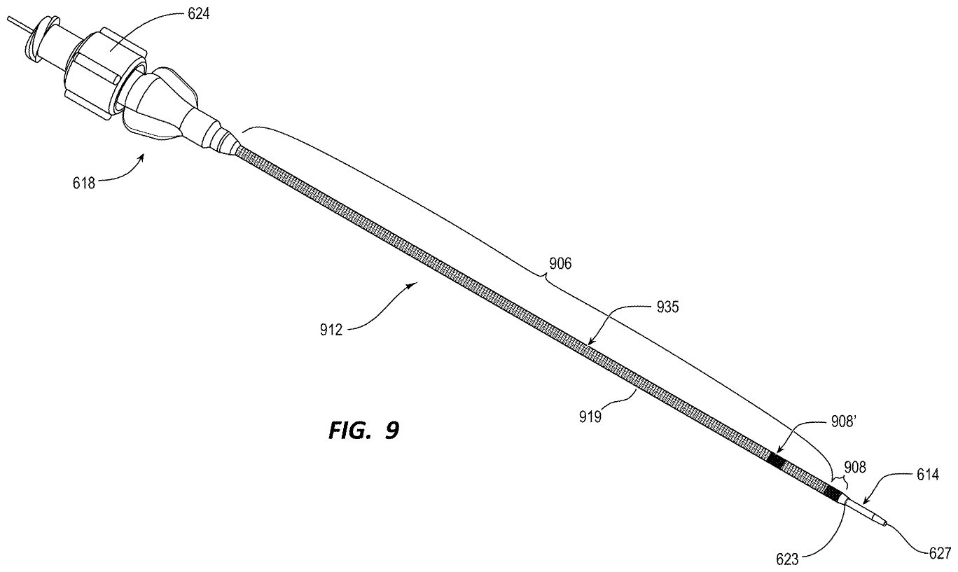

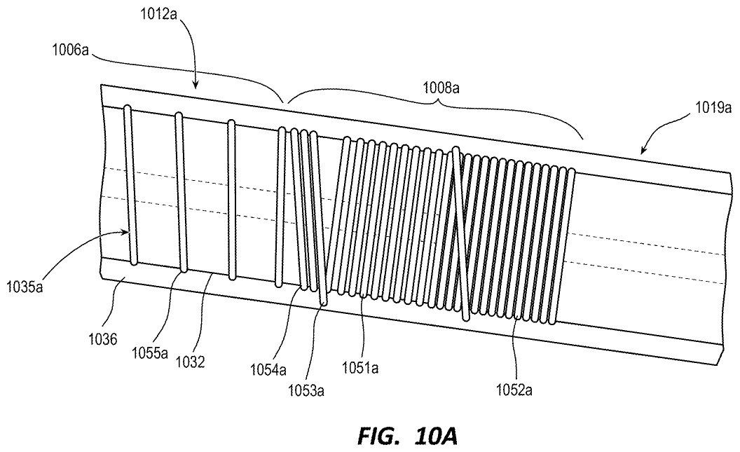

Turning now to FIG. 9 a perspective view of an introducer sheath assembly 900 formed according to embodiments of either the method 700 or the method 800 is shown. The introducer sheath assembly may include a catheter sheath 912, a dilator 914, and a guidewire (not visible). The catheter sheath 912 includes a sheath tube 919 with a coiled wire frame 935 disposed therein. For example, the catheter sheath 912 may include the coiled wire frame 935 disposed between a liner and a jacket. Unless otherwise noted, the coiled wire frame 935 may include any aspect (e.g. materials or shape) of the braided wire frame 134, 534, 635 or the coiled wire frame 234, 1035a, 1035c, 1135, and/or described in the methods 300, 400. Furthermore, the liner and the jacket of the catheter sheath 912 may include any aspect (e.g., materials or shape) of the liner 132, 232 and/or the jacket 136, 236, and/or described in the methods 300, 400.

In some embodiments, the coiled wire frame 935 includes wire disposed more densely in the distal portion 908 and an additional portion 908′ than the proximal portion 906 of the wire frame 935. The distal portion 908 of the wire frame 935 may include at least some wire coiled over other, more loosely coned wire. The wire frame 935, including the distal portion 908 and the proximal portion 906, may include, any aspect of other wire frames described herein. The introducer sheath assembly 900 also may include a safety line 101 or 201.

Returning to FIG. 7, a flow diagram of the method 700 of manufacturing or forming a sheath is shown, such as the sheath tube 919. Any of the introducer sheaths 100, 200, 500, 600, 900 described herein may be adapted to include a sheath formed according to the method 700. One or more steps shown in FIG. 7 may be optional and not necessarily included in each embodiment or process of the method 700. Further, various steps may be completed in different sequences from those shown in FIG. 7.

In the illustrated method 700, a liner having a proximal region and distal region is provided 707. The liner may include any liner described herein and/or formed according to any process described herein. The liner may be slid 708 over a PTFE coated mandrel, and an FEP tube may be slid 709 onto a proximal end of the liner on the mandrel. In embodiments having a thermoplastic inner lumen in the liner, the mandrel may be coated with the PTFO to prevent sticking to a non-coated wire. In some embodiments, the inner lumen of the liner may include a polyether block amide (e.g. PEBAX®). In some embodiments, the liner may be cut on the mandrel and the liner may be stretched after cutting to tighten the liner on the mandrel.

The method 700 may then include forming the coiled wire frame over the liner. The wire may include any wire described herein, such as a tungsten wire. A first (e.g. initial) portion of wire may then be coiled 710 over a first portion of the liner at least partially between the proximal region and the distal region. In act 710, the first portion of the wire may be coiled at a first coil pitch. A second portion of the wire may then be coiled 711 over the distal region of the liner to form a distal end of the coiled wire frame. The second portion of the wire may be coiled at a second coil pitch that is tighter (e.g., less distance between coils) than the first coil pitch in the act 710. A third portion of the wire may then be coiled 712 from the distal end of the coiled wire frame towards the proximal region of the liner and at least partially over the second portion of the wire. Accordingly, the third portion of the wire doubles back over at least some of the second portion of the wire. The third portion of the wire may be coiled with a third coiled pitch that is tighter than at least the first coil pitch in the act 710. In some embodiments, the third coil pitch is tighter than the second coil pitch.

A fourth portion of the wire may then be coiled 713 over at least some of the first (e.g., initial) portion and/or the second portion of the wire between the distal end of the coiled wire frame and the proximal region of the liner. In some embodiments, the fourth portion may be coiled over approximately a transition from the first (e.g., initial) portion to the second portion of the wire. The fourth portion of the wire may coiled be with a fourth coil pitch that is tighter than the first pitch in the act 710. In some embodiments, the fourth coil pitch is tighter than the second coil pitch and/or the third coil pitch. In relation to FIG. 9, the second portion, the third portion, and the fourth portion of the wire may together form the distal portion 908 of the coiled wire frame 935 having wire disposed more densely therein.

In some embodiments, the acts 712 and 713 are combined such that the third portion of the wire is coiled from the distal end of the coiled wire frame towards the proximal region of the liner and over the second portion of the wire and at least some of the first portion of the wire between the distal end of the coiled wire frame and the proximal region of the liner. The coil pitch of such a coiled wire in combining the acts 712 and 713 may be the fourth coil pitch that is tighter than the first coil pitch and/or at least as tight as or tighter than the second coil pitch. In some embodiments, an additional portion of wire is coiled over at least some of the second portion of the wire and between the third portion of the wire coiled at the third coil pitch of the act 712 and the fourth portion of the wire coiled at the fourth coil pitch of the act 713. This additional portion of the wire may be coiled at an additional coil pitch that is looser than both the third coil pitch and the fourth coil pitch.

The method 700 may then include at least some (e.g., all) of the first portion of the wire being removed 714. More specifically the segment of the first portion of the wire that is disposed between the fourth portion of the wire and the proximal region of the liner may be removed. Upon removal of at least some of the first portion of the wire, the segment of the first portion and/or the second portion of the wire that is under the fourth portion of the wire remains secured to the liner due to the tightness of fourth coil pitch coiled over that segment of the first portion and/or the second of the wire. Removing at least some of the first portion of the wire may include breaking, kinking, cutting or otherwise separating the segment of the first portion of the wire that is disposed between the fourth portion of the wire and the proximal region of the liner.

The method 700 may then include a fifth portion of the wire coiled 715 from the fourth portion of the wire to the proximal region of the liner. The fifth portion of the wire may be coiled at a fifth coiled pitch that is tighter than the first pitch and/or looser than the second pitch and the fourth pitch. Relating to FIG. 9, the fifth portion of the wire may formed the proximal portion 906 of the coiled wire frame 935 having wire disposed less densely therein than the distal portion 908. As the coiled wire frame doubles over at least a portion of itself, the wire in the coiled wire frame may be continuous from where the wire was removed, through the second portion, the third portion, the fourth portion, and the fifth portion of the wire in the coiled wire frame.

In some embodiments, the method 700 may include a sixth portion of the wire coiled between two regions of the fifth portion of the wire and spaced from the second portion of the wire. The sixth portion of the wire may be coiled at a sixth coil pitch that is tighter than the fifth pitch. In some embodiments, the sixth coil pitch is substantially the same as or similar to the second coil pitch and/or the fourth coil pitch. Relating to FIG. 9, the sixth portion of the wire may form the additional portion 908′ of the coiled wire frame 935 spaced from the distal portion 908 and having the wire disposed more densely therein than the proximal portion 906.

The method 700 may then include securing the jacket over the coiled wire frame and the liner. For example, the FEP of step 709 may be slid 716 over some of the fifth portion of the wire of step 715, and the FEP may be heat shrunk 717 over some of the fifth portion of the wire. A jacket may be slid 718 over the liner including coiled wire frame, and FEP may be slid 719 at least partially over the jacket. The jacket may include any jacket described herein, and the FEP in act 719 may include the same or different FEP as the FEP in the acts 709 and 716. The FEP and jacket may be heat shrunk 720 over the coiled wire frame and the liner, and the FEP may be compressed 721. The liner and the jacket may be reflowed 722 before the assembly is removed 723 from the mandrel and the FEP is removed 724. In some embodiments, securing the jacket over the coiled wire frame and the liner may include any of the acts 312-322 described in relation to the method 300 and/or the acts 412-422 described in relation to the method 400.

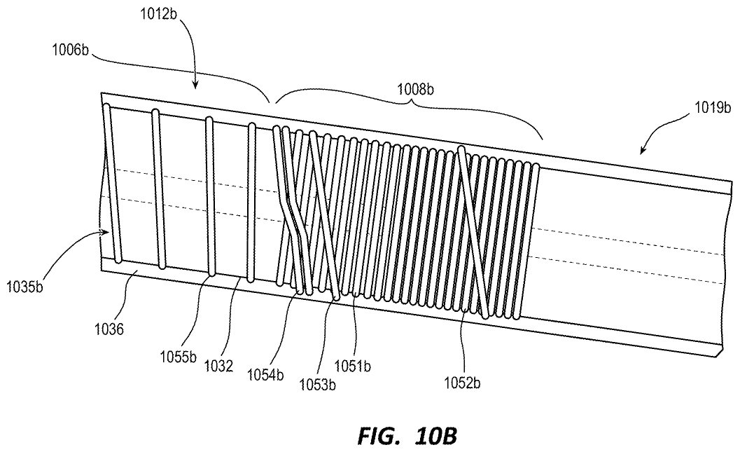

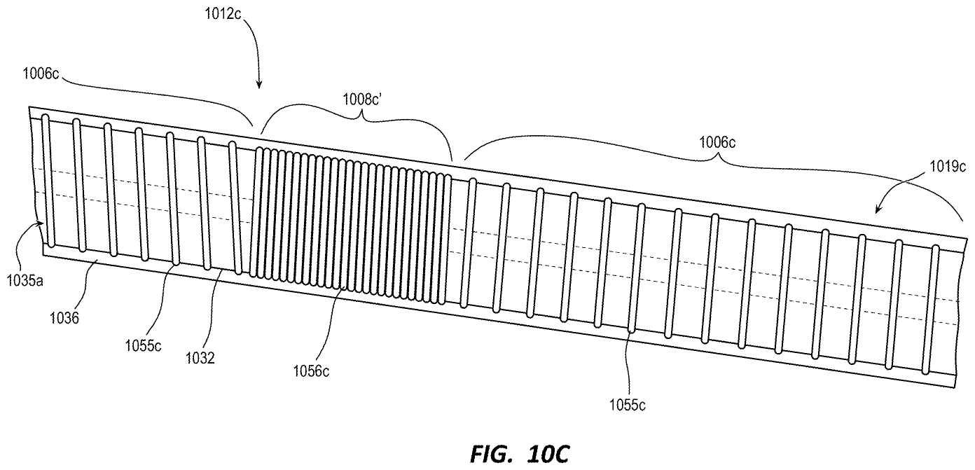

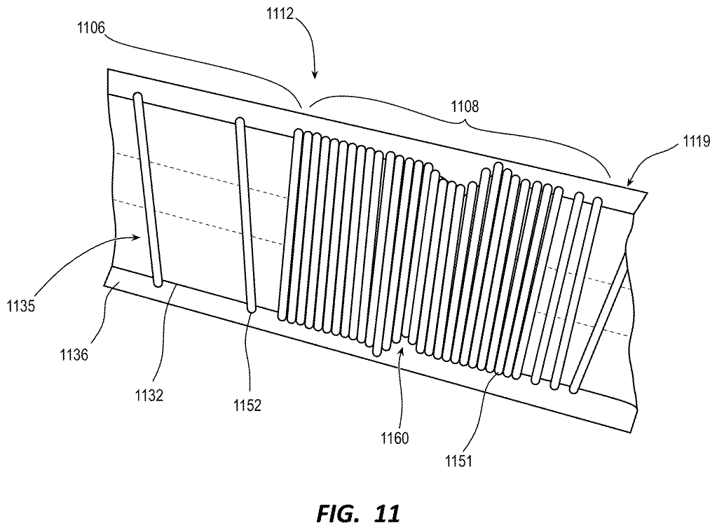

Turning ahead in the drawings to FIGS. 10A-10C, portions of a sheath formed according to one or more embodiments of the method 700 are shown. With specific reference to FIG. 10A, a portion of a sheath tube 1019a of a catheter sheath 1012a is shown. The sheath tube 1019a may be utilized with or adapted to any of the introducer sheaths described herein. The sheath tube 1019a may include a jacket 1036 and a liner 1032. The jacket 1036 and the liner 1032 may include any liner and jacket described herein.

The sheath tube 1019a also may include a coiled wire frame 1035a disposed between the liner 1032 and the jacket 1036. The coiled wire frame 1035a may include a proximal portion 1006a (similar or the same as the proximal portion 906) having a first portion 1055a wire coiled around the liner at a first coil pitch. (The first portion 1055a of wire in FIG. 10A coiled around the liner at the first coil pitch may correspond to the fifth portion of wire in the method 700 coiled around the liner from the fourth portion of the wire towards the proximal region at the fifth coil pitch of act 715.)

The coiled wire frame 1035a also may include a distal portion 1008a (similar to the distal portion 908) having the wire disposed more densely therein than the proximal portion 1006a of the coiled wire frame 1035a. In some embodiments, the distal portion 1008a of coiled wire frame 1035a includes a second portion 1051a of the wire coiled at a second pitch (and corresponding to the second portion of wire in the method 700 coiled around the distal end region with a second coil pitch) that is tighter than the first pitch.

The distal portion 1008a of coiled wire frame 1035a also may include a third portion 1052a of the wire coiled at a third coiled pitch over at least some of the second portion of the wire and the distal end region 1008a (and corresponding to the third portion of the wire in the method 700 coiled from the distal end towards the proximal region at least partially over the distal region and the second portion of the wire). The third coil pitch of the third portion 1052a of the wire may be tighter than the second coil pitch of the second portion 1051a of the wire of the coiled wire frame 1035a. In many embodiments, the third coil pitch of the third portion 1052 of the wire may be tighter than the first coil pitch of the first coil portion 1055a of the coiled wire frame 1035a.

The distal portion of the coiled wire frame 1035a may include a fourth portion 1054a of the wire coiled at a fourth coil pitch tighter than the first coil pitch in the first portion 1055a of the wire in the proximal region 1006a. (The fourth portion 1054a of the wire coiled at the fourth coil pitch may correspond to the fourth portion of the wire coiled at the fourth coil pitch in the method 700). The fourth coil pitch of the fourth portion 1054a of the wire may be as tight as or tighter than the third coil pitch of the third portion 1052a of the wire. In some embodiments, an additional portion 1053a of wire is coiled over at least some of the second portion 1051a of the wire and between the third portion 1052a of the wire and the fourth portion 1054a of the coiled wire frame 1035a. This additional portion 1053a of the wire may be coiled at an additional coil pitch that is looser than both the third coil pitch and the fourth coil pitch. The first portion 1055a of the wire may extend from the fourth portion 1054a of the wire towards the proximal end region of the sheath tube 1019a. The first portion 1055a, the second portion 1051a, the second portion 1052a, the additional portion 1053a, and the fourth portion 1054a may all be formed form a single, continuous wire.

Turning to FIG. 10B, the method 700 described at least some (e.g., all) of the first portion of the wire (not visible in FIG. 10B) being removed in act 714. More specifically, in the method 700, the segment of the first portion of the wire that is disposed between the fourth portion of the wire and the proximal region of the liner may be removed. In FIG. 10B a region 1060 of the fourth portion 1054a of the wire indicates from the initial (first) portion of the wire in the method 700 was removed. As shown in FIG. 10B, the second portion 1051a begins under this region 1060 of the fourth portion 1054a of the wire such that the fourth portion 1054a of the wire holds down the second portion 1051a and prevents the second portion 1051a of the wire from uncoiling.

Turning now to FIG. 10C, a portion of a sheath tube 1019c of a catheter sheath 1012c is shown. The sheath tube 1019c may be utilized with or adapted to any of the introducer sheaths described herein, including the sheath tube 1019a. The sheath tube 1019c may include the jacket 1036 and the liner 1032. The jacket 1036 and the liner 1032 may include any liner and jacket described herein.

The sheath tube 1019c also may include a coiled wire frame 1035c disposed between the liner 1032 and the jacket 1036. The coiled wire frame 1035c may include a proximal portion 1008a having a first portion 1055c of wire coiled around the liner 1032 at a first coil pitch, similar to or the same as the first coil pitch of the first portion 1055a of wire in the sheath tube 1019a. Though not visible in FIG. 10C, the coiled wire frame 1035c of the sheath tube 1019c also may include a wire frame 1035a also may include the distal portion 1008a having the wire disposed more densely therein than the proximal portion 1006a of the coiled wire frame 1035a. In some embodiments, the wire frame 1035c includes an additional portion 1008c′ (similar to the additional portion 908′) of the wire coiled between two regions of the first portion 1006c of the wire and coiled tighter than the first pitch. This additional portion 1008c′ of the wire is spaced from the distal portion 1008a (not visible in FIG. 10C).

Turning back to FIG. 8, a flow diagram of the method 800 of manufacturing or forming a sheath is shown, such as the sheath tube 919. Any of the introducer sheaths 100, 200, 500, 600, 900 described herein may be adapted to include a sheath formed according to the method 800. One or more steps shown in FIG. 8 may be optional and not necessarily included in each process. Further, various steps may be completed in different sequences from those shown in FIG. 8.

In the illustrated method 800 and similar to the method 700 of FIG. 7, a liner having a proximal region and distal region is provided 707, the liner may be slid 708 over a PTFE coated mandrel, and an FEP tube may be slid 709 onto a proximal end of the liner on the mandrel.

The method 800 may then include forming the coiled wire frame over the liner. The wire may include any wire described herein, such as a tungsten wire. The method 800 may include a first (e.g., initial) portion of wire coiled 810 over a first portion of the liner at least partially between the proximal region and the distal region to form a distal end of the coiled wire frame. The first portion of the wire may be coiled at a first coil pitch.

The method 800 may then include a second portion of the wire coiled 811 from the distal end of the coiled wire frame towards the proximal region of the liner and at least partially over the first portion of the wire. Accordingly, the second portion of the wire doubles back over at least some of the first (e.g., initial) portion of the wire. The second portion of the wire may coiled with a second coil pitch that is tighter than the first coil pitch.

The method 800 may then include removal 812 of at least some of the first (e.g., initial) portion of the wire. More specifically the segment of the first portion of the wire that is disposed between the second portion of the wire and the proximal region of the liner may be removed. Upon removal of some of the first portion of the wire, the segment of the first portion of the wire that is under the second portion of the wire remains secured to the liner due to the tightness of second coil pitch coiled over that segment of the first portion of the wire. Removing at least some of the first portion of the wire may include breaking, kinking, cutting or otherwise separating the segment of the first portion of the wire that is disposed between the second portion of the wire and the proximal region of the liner.

The method 800 may then include a third portion of the wire coiled 813 from the second portion of the wire to the proximal region of the liner at a third pitch that is tighter than the first pitch and looser than the second pitch. The segment of the first portion of wire covered by the second portion of wire, the second portion of wire, and the third portion of wire may be continuous with one another. In some embodiments of the method 800, a fourth portion of the wire is coiled between two regions of the third portion of the wire and spaced from the second portion of the wire. The fourth portion of the wire may be coiled at a fourth pitch that is tighter than the third pitch.

The method 800 may then include securing the jacket over the coiled wire frame and the liner. For example, the FEP of step 709 may be slid 814 over some of the third portion of the wire of step 813, and the FEP may be heat shrunk 815 over some of the third portion of the wire. Similar to the method 700 of FIG. 7, a jacket may be slid 718 over the liner including coiled wire frame, and FEP may be slid 719 at least partially over the jacket in the method 800. The jacket may include any jacket described herein, and the FEP in act 719 may include the same or different FEP as the FEP in the acts 709 and 716. The FEP and jacket may be heat shrunk 720 over the coiled wire frame and the liner, and the FEP may be compressed 721 in the method 800. The liner and the jacket may be reflowed 722 before the assembly is removed 723 from the mandrel and the FEP is removed 724 in the method 800. In some embodiments of the method 800, securing the jacket over the coiled wire frame and the liner may include any of the acts 312-322 described in relation to the method 300 and/or the acts 412-422 described in relation to the method 400.

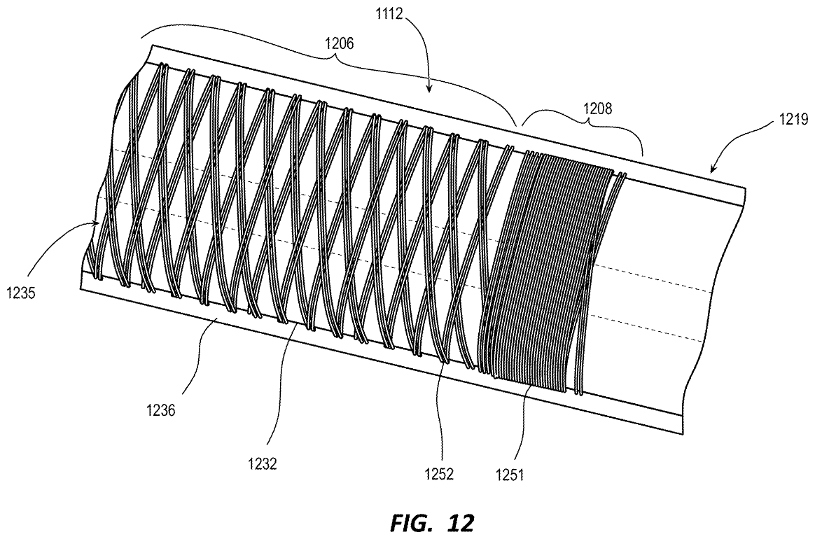

Turning ahead in the drawings to FIG. 11, a portion of a sheath formed according to one or more embodiments of the method 800 are shown. For example, a sheath tube 1119 of a catheter sheath 1112 may be utilized with or adapted to any of the introducer sheaths described herein. The sheath tube 1119 may include a jacket 1136 and a liner 1132. The jacket 1136 and the liner 1132 may include any liner and jacket described herein.

The sheath tube 1119 also may include a coiled wire frame 1135 disposed between the liner 1132 and the jacket 1136. The coiled wire frame 1135 may include a proximal portion 1106 (similar to the proximal portion 906) having a first portion 1152 of wire coiled around the liner 1132 at a first coil pitch. (The first portion 1152 of wire in FIG. 11 coiled around the liner at the first coil pitch may correspond to the third portion of wire in the method 800 coiled around the liner from the second portion of the wire towards the proximal region at the third coil pitch of act 813.)

The coiled wire frame 1135 also may include a distal portion 1108 (similar to the distal portion 908) having the wire disposed more densely therein than the proximal portion 1106 of the coiled wire frame 1135. In some embodiments, the distal portion 1108 of coiled wire frame 1135 includes a second portion 1151 of the wire coiled at a second pitch (and corresponding to the second portion of wire in the method 800 coiled around the distal end region with a second coil pitch) that is tighter than the first coil pitch. The first portion 1152 of the wire may extend from second portion 1151 of the wire towards the proximal end region. Accordingly, the first portion 1152 of the wire may be continuous with the second portion 1151 of the wire.

The second portion 1151 of the wire also may be coiled over a segment of wire coiled around the liner (corresponding to the first portion of the wire coiled around the liner in the method 800). As described in the method 800, at least some (e.g., all) of the initial (first) portion of the wire in the method 800 (not visible in FIG. 11) may be removed in act 812. More specifically, in the method 800, the segment of the initial (first) portion of the wire that is disposed between the second portion of the wire and the proximal region of the liner may be removed. In FIG. 11 a region 1160 of the second portion 1151 of the wire indicates from the initial (first) portion of the wire in the method 800 is covered by the second portion 1151 of the wire. As shown in FIG. 11, the second portion 1151 doubles back over the initial portion of the wire to cover the initial portion of the wire coiled over the liner such that the second portion 1151 of the wire holds down the initial portion and prevents the wire from uncoiling.

Turning now to FIG. 12, aspects of the braided wire frames described herein may be combined with aspects of the coiled wire frames described herein. A sheath tube 1219 of a catheter sheath 1212 may be utilized with or adapted to any of the introducer sheaths described herein. The sheath tube 1219 may include a jacket 1236 and a liner 1232. The jacket 1236 and the liner 1232 may include any liner and jacket described herein.

The sheath tube 1219 also may include a wire frame 1235 disposed between the liner 1232 and the jacket 1236. The coiled wire frame 1235 may include a braided proximal portion 1206 having a first portion 1252 of wire braided around the liner 1232. The braid of the first portion 1252 may include any aspect of braided wire frames described herein.

The coiled wire frame 1235 also may include a distal portion 1208 (similar to the distal portion 908) having a second portion of wire 1251 coiled around the liner 1232 and disposed more densely than the first portion 1252 of the braided wire in the proximal portion 1206. In some embodiments, the second portion 1251 of wire coiled around the liner 1232 is also coiled around a distal end of the first portion 1252 of the wire braided around the liner 1232. Coiling the second portion 1251 around the distal end of the first portion 1252 of the wire braided around the liner 1232 may prevent the braided wire from springing open, even after the jacket 1236 is heated. In some embodiments, at least one or both of the second portion 1251 and the first portion 1252 may be annealed during formation of the sheath, as described elsewhere herein.

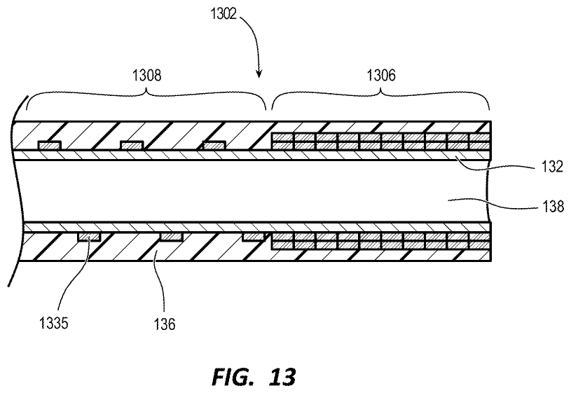

As provided above, some embodiments of sheaths disclosed herein may comprise wire frame having a flat wire. Turning to FIG. 13, a cross-sectional view of a portion of a sheath shaft 1302 is shown. Unless otherwise noted, the sheath shaft 1302 may include any aspect of sheaths described herein, and may be formed according to any of the methods described herein. In some embodiments, the sheath shaft 1302 may include coiled flat wire forming a wire frame 1335, a liner 132 including the sheath shaft lumen 138, and a jacket 136. The wire frame 1335 may comprises a proximal portion 1308 and a distal portion 1306 having the flat wire disposed more densely therein than the proximal portion 1308 of the wire frame 1335. In particular, the flat wire of the wire frame 1335 may allow the flat wire to be more densely packed in the distal portion 1306 than round wire. For example, sides of the flat wire of the wire frame 1335 may more uniformly abut sides of adjacent flat wire in the distal portion 1335, resulting in the distal portion 1306 having improved fluoroscopic identification. In some embodiments, the wire frame 1335 may include at least an inner layer of the flat wire and an outer layer of the flat wire coiled around substantially all of the inner layer of the flat wire in the distal portion 1306. While the sheath shaft 1302 of FIG. 13 includes two layers of flat wire (e.g., one layer wrapped over another layer) in the distal portion 1306 of the wire frame 1335, in some embodiments, the distal portion 1306 may include only a single layer of the flat wire, with the sides or edges of the flat wire abutting sides or edges of adjacent flat wire(s) in the distal portion 1306.

### EXAMPLES

The following examples are potential combinations and embodiments within the scope of the present disclosure. Recitation of these examples are further illustrations of embodiments and are not limiting to the rest of the disclosure in any way. These examples are additions to embodiments and descriptions found throughout this disclosure are not to be understood as limiting any of the preceding examples, embodiments, or disclosure in any way. Moreover, these examples are not to be understood as limiting or changing the scope of the claims in any way.

These examples include:

An introducer sheath comprising: a shaft forming a lumen and comprising: a braided or coiled wire frame having a distal portion and a proximal portion, wherein a wire is disposed more densely in the distal portion than the proximal portion of the wire frame; a jacket encompassing the wire frame, and a liner forming an inner wall, the liner encompassed by the wire frame.

An introducer sheath, wherein a distal portion having a wire disposed more densely therein than a proximal portion of a wire frame has a longitudinal length of less than about 125 mm.

An introducer sheath, wherein a wire frame includes a radiopaque material.

An introducer sheath, wherein a wire frame includes a coiled wire frame and a distal portion of the coiled wire frame includes a first portion of coiled wire extending a length of the distal portion and a second portion of coiled wire extending the length of the distal portion such that the second portion of coiled wire overlaps the first portion of coiled wire and the wire of the distal portion is more densely disposed than the proximal portion of the wire frame.

An introducer sheath, wherein: a first portion of coiled wire extends from a first end of a distal portion of a coiled wire frame to a distal end of the distal portion; and a second portion of coiled wire is connected to the first portion of coiled wire at the distal end and extends from the distal end to the first end of the distal portion, the second portion of coiled wire having a terminating end at the first end of the distal portion of the coiled wire frame.

An introducer sheath, wherein a coiled wire frame includes a round wire.

An introducer sheath, wherein a coiled wire frame is free from annealing.

An introducer sheath, wherein a coiled wire frame includes a flat wire.

An introducer sheath, wherein a wire frame includes a braided wire frame, wherein a distal portion of the braided wire frame has more picks/inch than a proximal portion of the braided wire frame such that a wire of the distal portion is more densely disposed than the proximal portion of the wire frame.

An introducer sheath, wherein a distal portion includes an annealed region preventing a braided wire frame from unraveling at a distal end during assembly, a proximal portion of the braided wire frame being unannealed and maintaining physical properties of spring temper wire.

An introducer sheath, comprising a safety line extending longitudinally through a wire frame and secured to a proximal portion and a distal portion of the wire frame.

An introducer sheath, wherein a safety line includes a single tungsten round wire.

An introducer sheath, wherein a safety line includes a plurality of aramid fibers.

An introducer sheath, wherein a braided or coiled wire frame comprises: a first intermediate portion disposed at least proximate to a distal portion; a second intermediate portion disposed between the first intermediate portion and a proximal portion, wherein a wire is disposed more densely in the second intermediate portion than the first intermediate portion and the proximal portion.

An introducer sheath, wherein a wire frame includes a coiled wire frame comprising: a proximal portion having a first portion of a wire coiled at a first pitch; and a distal portion having a second portion of the wire coiled at a second pitch that is tighter than the first pitch, a third portion of the wire coiled at a third pitch over at least some of the second portion of the wire, and a fourth portion of the wire coiled at a fourth pitch tighter than the first pitch, the first portion of the wire extending from the fourth portion of the wire towards the proximal portion.

An introducer sheath, wherein a wire frame includes a coiled wire frame comprising: a proximal portion having a first portion of a wire coiled at a first pitch; and a distal portion having a second portion of the wire coiled at a second pitch that is tighter than the first pitch and coiled over a segment of the wire coiled around a liner, the first portion of the wire extending from the second portion of the wire towards the proximal portion.

An introducer sheath, wherein a wire frame includes a coiled wire frame comprising a portion of a wire disposed between two regions of a first portion of the wire and coiled tighter than a first pitch.

An introducer sheath, wherein a wire frame includes: at least a proximal portion of a wire frame comprises braided wire; a distal portion of the wire frame comprises coiled wire that is coiled over at least a portion of the braided wire such that the wire of the wire frame is disposed more densely in the distal portion than the proximal portion.

An introducer sheath, wherein a wire frame includes a substantially flat wire coiled more densely in a distal portion than the proximal portion of the wire frame with sides of the flat wire in the distal portion abutting adjacent flat wires of the wire frame in the distal portion, the wire frame including at least an inner layer of the flat wire and an outer layer of the flat wire coiled around substantially all of the inner layer of the flat wire in the distal portion.

An introducer sheath, comprising a hub forming a chamber and comprising: a side port in fluid communication with the chamber, and an introducer bore with a seal to maintain hemostasis of the introducer sheath while allowing a medical instrument to be introduced through the introducer bore into the chamber, wherein the shaft is coupled to the hub at a proximal end of the shaft, the lumen of the shaft being in fluid communication with the chamber of the hub.

An introducer sheath, comprising a dilator positionable internal to, and along the length of a shaft, the dilator comprising: a stiffener tube having a proximal end connected to a dilator hub and a distal end; and a sleeve positioned external to the stiffener, a distal end of the sleeve extending beyond the distal end of the stiffener and adapted for form a dilator tip; wherein the distal end of the stiffener is configured to be positioned inside the shaft, a portion of the dilator tip is positioned inside the shaft, and a portion of the dilator tip is positioned outside the shaft such that the shaft provides strain relief subsequent to lateral movement of the dilator tip to prevent kinking of the dilator tip at the transition between the stiffener tip and the dilator tip.

A method of manufacturing an introducer sheath, the method comprising: forming a braided and/or coiled wire frame of a sheath shaft having a distal portion and a proximal portion, wire being disposed more densely in the distal portion than in the proximal portion; reflowing a liner to an interior surface of the wire frame; reflowing a jacket to an exterior surface of the wire frame; and securing a hub to the proximal portion of the wire frame, the hub having a chamber in fluid communication with a hollowing conduit formed from reflowing the liner and the jacket to the wire frame, a side port in fluid communication with the chamber, and an introducer bore with a seal to maintain hemostasis of the introducer sheath while allowing a medical instrument to be introduced through the introducer bore into the chamber.

A method of manufacturing an introducer sheath, comprising forming a braided or coiled wire frame having a distal portion and a proximal portion with wire disposed more densely in the distal portion than in the proximal portion and the distal portion having a longitudinal length of less than about 125 mm.

A method of manufacturing an introducer sheath, comprising forming a coiled wire frame by winding wire on a spring winder from a proximal portion to a distal portion and then winding the wire over the distal portion again such that the wire is disposed more densely in the distal portion than in the proximal portion.

A method of manufacturing an introducer sheath, wherein a wire extends continuously from a proximal portion to a distal end of a distal portion opposite to the proximal portion and from the distal end to a terminating end of the wire at a first end of the distal portion between the distal end and the proximal portion.

A method of manufacturing an introducer sheath, wherein a wire includes a round wire and a coiled wire frame is free from annealing.

A method of manufacturing an introducer sheath, comprising braiding metal wires at a first picks/inch to form a proximal portion of a wire frame and braiding the metal wires at a second picks/inch that is greater than the first picks/inch to form a distal portion having the wire disposed more densely than in the proximal portion.

A method of manufacturing an introducer sheath, comprising annealing an end region of a distal portion to prevent a wire frame from unraveling at an end region during assembly, and overmolding a hub around at least some of a proximal portion of the wire frame to prevent the wire frame from unraveling at a proximal end.

A method of manufacturing an introducer sheath, comprising adjoining a tip to a distal end at of the distal portion of a wire frame.

A method of manufacturing an introducer sheath, comprising disposing an axial fiber longitudinally through a wire frame and securing the axial fiber to a proximal portion and a distal portion of the wire frame before reflowing a liner and reflowing a jacket.

A method of manufacturing an introducer sheath, comprising braiding wires to form at least a proximal portion of a wire frame; and coiling wire over at least some of the braided wires to form a distal portion of the wire frame having the wire of the wire frame disposed more densely than the proximal portion.

A method of manufacturing an introducer sheath, the method comprising: extruding a nylon core; braiding or coiling wire over the nylon core to form a wire frame having a distal portion and a proximal portion, the wire being disposed more densely in the distal portion than in the proximal portion; heating and stretching the nylon core to reduce the diameter of the nylon core for removal of the wire frame; reflowing a liner to an interior surface of the wire frame and a jacket to an exterior surface of the wire frame to form a composite conduit; cutting the composite conduit proximate to the proximal portion and the distal portion of the wire frame to form a sheath shaft; and securing a hub to the sheath shaft proximate to the proximal portion of the wire frame.

A method of manufacturing an introducer sheath, the method comprising: providing a liner having a proximal region and a distal region; forming a coiled wire frame over the liner, including: coiling wire on the liner; coiling the wire over at least some of the wire at the distal region; removing at least some of the wire coiled on the liner between the distal region and the proximal region; and coiling the wire from the distal region towards the proximal region at a pitch that is looser than the wire coiled at the distal region; and securing a jacket over the coiled wire frame and the liner.

A method of manufacturing an introducer sheath, comprising: coiling a first portion of wire over a first portion of a liner at least partially between a proximal region and a distal region, the first portion of the wire being coiled at a first pitch; coiling a second portion of the wire over the distal region of the liner to form a distal end of a coiled wire frame, the second portion of the wire being coiled at a second pitch that is tighter than the first pitch; coiling a third portion of the wire from the distal end of the coiled wire frame towards the proximal region of the liner and at least partially over the second portion of the wire, the third portion of the wire being coiled with a third pitch that is tighter than the first pitch; coiling a fourth portion of the wire over at least some of the first portion of the wire between the distal end of the coiled wire frame and the proximal region of the liner, the fourth portion of the wire being coiled with a fourth pitch that is tighter than the first pitch; removing the first portion of the wire disposed between the fourth portion of the wire and the proximal region of the liner; and coiling a fifth portion of the wire from the fourth portion of the wire to the proximal region of the liner at a fifth pitch that is tighter than the first pitch and looser than the second pitch and the fourth pitch.

A method of manufacturing an introducer sheath, comprising: coiling a sixth portion of wire between two regions of a fifth portion of the wire and spaced from a second portion of the wire, the sixth portion of the wire being coiled at a sixth pitch that is tighter than a fifth pitch.

A method of manufacturing an introducer sheath, comprising: coiling a first portion of wire over a first portion of a liner at least partially between a proximal region and a distal region to form a distal end of a coiled wire frame, the first portion of the wire being coiled at a first pitch; coiling a second portion of the wire from the distal end of the coiled wire frame towards the proximal region of the liner and at least partially over the first portion of the wire, the second portion of the wire being coiled with a second pitch that is tighter than the first pitch; removing the first portion of the wire disposed between the second portion of the wire and the proximal region of the liner; and coiling a third portion of the wire from the second portion of the wire to the proximal region of the liner at a third pitch that is tighter than the first pitch and looser than the second pitch.

A method of manufacturing an introducer sheath, comprising: coiling a fourth portion of wire between two regions of a third portion of the wire and spaced from a second portion of the wire, the fourth portion of the wire being coiled at a fourth pitch that is tighter than a third pitch.

A method of manufacturing an introducer sheath, comprising, before securing a jacket over a coiled wire frame and a liner: sliding a heat shrink material over at least some of a wire coiled at a proximal region of the liner; and heat shrinking the heat shrink material over the at least some of the wire coiled at the proximal region of the liner.

A method of manufacturing an introducer sheath, comprising: sliding a jacket over a coiled wire frame; and heat shrinking the jacket over the coiled wire frame.

A method of manufacturing an introducer sheath, comprising reflowing a liner and a jacket after securing the jacket over a coiled wire frame and the liner.

References to approximations are made throughout this specification, such as by use of the term “near.” For each such reference, it is to be understood that, in some embodiments, the value, feature, or characteristic may be specified without approximation. For example, where qualifiers such as “near” and “approximately” are used, these terms include within their scope the qualified words in the absence of their qualifiers. For example, where the term “approximately aligned” is recited with respect to a feature, it is understood that in further embodiments, the feature can have a precisely aligned configuration.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. An introducer sheath comprising:
a shaft forming a lumen and comprising:
a braided or coiled wire frame having a distal portion and a proximal portion, wherein a wire is disposed more densely in the distal portion than the proximal portion of the wire frame;
a jacket encompassing the wire frame, and
a liner forming an inner wall, the liner encompassed by the wire frame.

2. The sheath of claim 1, wherein the wire frame includes a coiled wire frame and the distal portion of the coiled wire frame includes a first portion of coiled wire extending a length of the distal portion and a second portion of coiled wire extending the length of the distal portion such that the second portion of coiled wire overlaps the first portion of coiled wire and the wire of the distal portion is more densely disposed than the proximal portion of the wire frame.

3. The sheath of claim 1, wherein the wire frame includes a braided wire frame, wherein the distal portion of the braided wire frame has more picks/inch than the proximal portion of the braided wire frame such that the wire of the distal portion is more densely disposed than the proximal portion of the wire frame.

4. The sheath of claim 1, further comprising a safety line extending longitudinally through the wire frame and secured to the proximal portion and the distal portion.

5. The sheath of claim 1, wherein the braided or coiled wire frame further comprises:
a first intermediate portion disposed at least proximate to the distal portion;
a second intermediate portion disposed between the first intermediate portion and the proximal portion, wherein the wire is disposed more densely in the second intermediate portion than the first intermediate portion and the proximal portion.

6. The sheath of claim 1, wherein the wire frame includes a coiled wire frame comprising:
the proximal portion having a first portion of a wire coiled at a first pitch; and
the distal portion having a second portion of the wire coiled at a second pitch that is tighter than the first pitch, a third portion of the wire coiled at a third pitch over at least some of the second portion of the wire, and a fourth portion of the wire coiled at a fourth pitch tighter than the first pitch, the first portion of the wire extending from the fourth portion of the wire towards the proximal portion.

7. The sheath of claim 1, wherein the wire frame includes a coiled wire frame comprising:
the proximal portion having a first portion of a wire coiled at a first pitch; and
the distal portion having a second portion of the wire coiled at a second pitch that is tighter than the first pitch and coiled over a segment of the wire coiled around the liner, the first portion of the wire extending from second portion of the wire towards the proximal portion.

8. The sheath of claim 1, wherein the wire frame includes:
at least the proximal portion of the wire frame comprises braided wire;
the distal portion of the wire frame comprises coiled wire that is coiled over at least a portion of the braided wire such that the wire of the wire frame is disposed more densely in the distal portion than the proximal portion.

9. The sheath of claim 1, wherein the wire frame includes a substantially flat wire coiled more densely in the distal portion than the proximal portion of the wire frame with sides of the flat wire in the distal portion abutting adjacent flat wires of the wire frame in the distal portion, the wire frame including at least an inner layer of the flat wire and an outer layer of the flat wire coiled around substantially all of the inner layer of the flat wire in the distal portion.

10. The sheath of claim 1, further comprising a hub forming a chamber and comprising:
a side port in fluid communication with the chamber, and
an introducer bore with a seal to maintain hemostasis of the introducer sheath while allowing a medical instrument to be introduced through the introducer bore into the chamber,
wherein the shaft is coupled to the hub at a proximal end of the shaft, the lumen of the shaft being in fluid communication with the chamber of the hub.

11. The sheath of claim 1, further comprising a dilator positionable internal to, and along the length of the shaft, the dilator comprising:
a stiffener tube having a proximal end connected to a dilator hub and a distal end; and
a sleeve positioned external to the stiffener, a distal end of the sleeve extending beyond the distal end of the stiffener and adapted for form a dilator tip;
wherein the distal end of the stiffener is configured to be positioned inside the shaft, a portion of the dilator tip is positioned inside the shaft, and a portion of the dilator tip is positioned outside the shaft such that the shaft provides strain relief subsequent to lateral movement of the dilator tip to prevent kinking of the dilator tip at the transition between the stiffener tip and the dilator tip.

12. A method of manufacturing an introducer sheath, the method comprising:
forming a braided and/or coiled wire frame of a sheath shaft having a distal portion and a proximal portion, wire being disposed more densely in the distal portion than in the proximal portion;
reflowing a liner to an interior surface of the wire frame;
reflowing a jacket to an exterior surface of the wire frame; and
securing a hub to the proximal portion of the wire frame, the hub having a chamber in fluid communication with a hollowing conduit formed from reflowing the liner and the jacket to the wire frame, a side port in fluid communication with the chamber, and an introducer bore with a seal to maintain hemostasis of the introducer sheath while allowing a medical instrument to be introduced through the introducer bore into the chamber.

13. The method of claim 12, wherein forming a braided or coiled wire frame includes forming a coiled wire frame by winding the wire on a spring winder from the proximal portion to the distal portion and then winding the wire over the distal portion again such that the wire is disposed more densely in the distal portion than in the proximal portion.

14. The method of claim 12, wherein forming a braided or coiled wire frame having a distal portion and a proximal portion includes braiding metal wires at a first picks/inch to form the proximal portion of the wire frame and braiding the metal wires at a second picks/inch that is greater than the first picks/inch to form the distal portion having the wire disposed more densely than in the proximal portion.

15. The method of claim 12, further comprising adjoining a tip to a distal end at of the distal portion of the wire frame.

16. The method of claim 12, further comprising disposing an axial fiber longitudinally through the wire frame and securing the axial fiber to the proximal portion and the distal portion before reflowing the liner and reflowing the jacket.

17. The method of claim 12, wherein forming a braided and/or coiled wire frame of a sheath shaft having a distal portion and a proximal portion includes:
braiding wires to form at least the proximal portion of the wire frame; and
coiling wire over at least some of the braided wires to form the distal portion having the wire of the wire frame disposed more densely than the proximal portion.

18. A method of manufacturing a sheath, the method comprising:
providing a liner having a proximal region and a distal region;
forming a coiled wire frame over the liner, including:
coiling wire on the liner;
coiling the wire over at least some of the wire at the distal region;
removing at least some of the wire coiled on the liner between the distal region and the proximal region; and
coiling the wire from the distal region towards the proximal region at a pitch that is looser than the wire coiled at the distal region; and
securing a jacket over the coiled wire frame and the liner.

19. The method of claim 18, wherein forming the coiled wire frame over the liner includes:
coiling a first portion of wire over a first portion of the liner at least partially between the proximal region and the distal region to form a distal end of the coiled wire frame, the first portion of the wire being coiled at a first pitch;
coiling a second portion of the wire from the distal end of the coiled wire frame towards the proximal region of the liner and at least partially over the first portion of the wire, the second portion of the wire being coiled with a second pitch that is tighter than the first pitch;
removing the first portion of the wire disposed between the second portion of the wire and the proximal region of the liner; and
coiling a third portion of the wire from the second portion of the wire to the proximal region of the liner at a third pitch that is tighter than the first pitch and looser than the second pitch.

20. The method of claim 18, further comprising, before securing the jacket over the coiled wire frame and the liner:
sliding a heat shrink material over at least some of the wire coiled at the proximal region of the liner; and
heat shrinking the heat shrink material over the at least some of the wire coiled at the proximal region of the liner.
